(19) United States
(12) Patent Application Publication
(10) Pub. No.: US 2004/0019091 A1
(43) Pub. Date: Jan. 29, 2004

(54) Triazole derivatives and pharmaceutical compositions comprising them

(76) Inventors: Eric Bignon, Pinsaguel (FR); Eva Csikos, Budapest (HU); Daniel Frehel, Estadens (FR); Csaba Gonczi, Budapest (HU); Gergley Heja, Budapest (HU); Miklos Morvai, Mogyorod (HU); Benjamin Podanyi, Budakeszi (HU); Erika Schlovicsko Varkonyine, (US)

Correspondence Address: SANOFI-SYNTHELABO INC., 9 GREAT VALLEY PARKWAY, P.O. BOX 3026, MALVERN, PA, 19355, US

(21) Appl. No.: 10/398,858

(22) PCT Filed: Oct. 25, 2001
(86) PCT No.: PCT/EP01/12984

(30) Foreign Application Priority Data
Oct. 26, 2000 (FR) 00/13728
Oct. 26, 2000 (HU) P 0004153

(51) Int. Cl. A61K 31/4196; C07D 249/14; C 07D 4 3/02

(52) U.S. Cl. 514/383; 548/265.4

(57) ABSTRACT

The present invention relates to compounds of formula (I) and their pharmaccutically acceptable salts, solvates, hydrates and polymorphs. These compounds are powerful and selective CCK₁ receptor agonists.

The present invention relates to novel triazole derivatives, to a process for preparing them and to pharmaceutical compositions comprising them.

These novel compounds are powerful and selective agonists of the CCK₁ (also called CCK-A) receptors of cholecystokinin (CCK).

CCK is a peptide which, in response to an ingestion of food, is secreted peripherally and participates in regulating many digestive processes (Crawley J. N. et al., Peptides, 1994, 15 (4), 731-735).

CCK has since been identified in the brain, and might be the most abundant neuropeptide acting as a neuromodulator of cerebral functions by stimulating CCK₂-type (also called CCK-B) receptors (Crawley J. N. et al., Peptides, 1994, 15 (4), 731-735). Within the central nervous system, CCK interacts with dopamine-mediated neuronal transmission (Crawley J. N. et al., ISIS Atlas of Sci., Pharmac, 1988, 84-90). It also plays a role in mechanisms involving acetylcholine, gaba (4-aminobutyric acid), serotonin, opioids, somatostatin and substance P and in ion channels. Its administration brings about physiological changes: palpebral ptosis, hypothermia, hyperglycaemia, catalepsis; and behavioural changes: hypolocomotion, reduction in exploration behaviour, analgesia, a change in learning faculty, and a change in sexual behaviour and satiety.

CCK exerts its biological activity via at least two types of receptor: CCK₁ receptors, located mainly peripherally, and CCK₂ receptors, essentially present in the cerebral cortex. The peripheral-type CCK₁ receptors are also present in certain regions of the central nervous system, including the postrema area, the solitary tract nucleus and the interpeduncular nucleus (Moran T. H. et al., Brain Research, 1986, 362, 175-179; Hill D. R. et al., J. Neurosci, 1990, 10, 1070-1081).

At the periphery, via CCK₁ receptors (Moran T. H. et al., Brain Research, 1986, 362, 175-179), CCK delays gastric drainage, modifies intestinal motility, stimulates vesicle contraction, increases bile secretion and controls pancreatic secretion (McHugh P. R. et al., Fed. Proc., 1986, 45, 1384-1390; Pendleton R. G. et al., J. Pharmacol. Exp. Ther., 1987, 241, 110-116).

The patent application WO 98/51686 describes a series of triazole derivatives possessing CCK₁ receptor agonist activity.

The present invention provides a 3-aminotriazole derivative of formula:

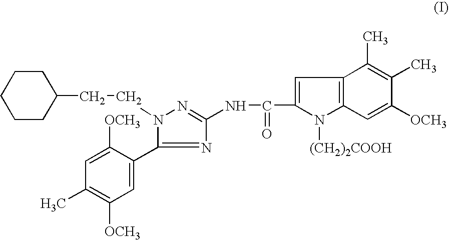

and its solvates, hydrates, polymorphs and pharmaceutically acceptable salts.

One specific aspect of the invention is constituted by compounds of formula (I) and the pharmaceutically acceptable salts thereof formed with organic or mineral bases, for example alkali metal or alkaline earth metal, such as sodium, potassium or calcium salts, or salts formed with an amine, such as trometanol, arginine or lysine. Another specific aspect of the invention is constituted by the polymorphic and solvate (pseudopolymorphic) forms of the 3-aminotriazole derivative of the formula (I), to the salts of the 3-aminotriazole derivative of the formula (I) and of its polymorphs and solvates, given with ethanolamine, diethanolamine, diethylamine or adamantanamine.

The 3-aminotriazole derivative of formula (I) falls under the general formula of the 3-aminotriazole derivatives described in patent application WO 98/51686, although, individually it has not been described.

The compound of formula (I), their solvates, polymorphs and salts are much more powerful CCK₁ agonists than those described in the prior art.

The compounds of the invention have indeed been the subject of studies for the purpose of characterizing:

their potentiality for displacing [¹²⁵I]-CCK from its binding sites present in rat pancreatic membranes (CCK₁ receptor) or 3T3 cells expressing recombinant human CCK₁ receptor;

their selectivity for the CCK₂ receptor;

their CCK₁ receptor agonist property, by way of their capacity to induce mobilization of intracellular calcium in vitro in 3T3 cells expressing the human CCK₁ receptor;

their agonist effect by the oral route on gastric drainage in the mouse.

These studies have shown that, in contrast to the compounds of the prior art, the compounds of the present invention surprisingly meet the various criteria below simultaneously: they possess not only a high affinity for CCK₁ receptors but also good selectivity for CCK₁ receptors (relative to CCK₂ receptors) and a powerful CCK₁ receptor agonist activity, demonstrated by the intracellular calcium mobilization and gastric drainage tests. These multiple properties make the compounds of the invention of major therapeutic interest as medicaments intended for the treatment of diseases which necessitate stimulation of CCK₁ receptors.

The compounds of the invention may be prepared in accordance with the methods described in the patent application WO 98/51686. Scheme 1 below illustrates their preparation method.

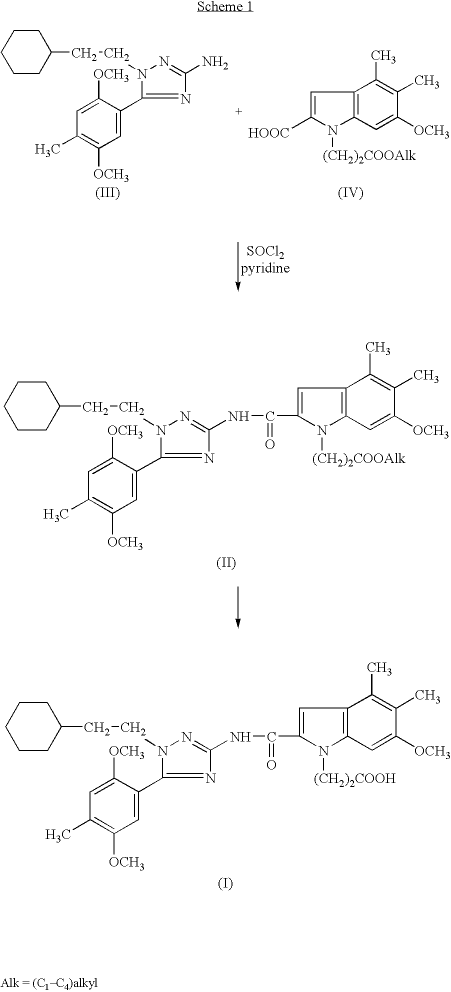

An other object of the present invention is the preparation process of compound of formula (I), its solvates, hydrates, polymorphs and pharmaceutically acceptable salts. This process is characterized in that:

a compound of formula:

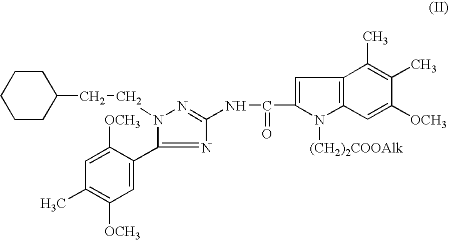

is hydrolysed;

If desired, the acid of formula (I) thus obtained is converted into its solvates, hydrates, polymorphs or pharmaceutical acceptable salts.

According to the preparation method the appropriate ester (II) is hydrolysed with a strong alkali and the acid of the formula (I) is liberated from the resulting salt, by using a strong mineral acid.

Surprisingly, depending on the conditions of the precipitation of the acid of the formula (I), on the temperature of the precipitation, on the addition rate of the acid, on the gradient of the cooling, on the rotation rate of the stirrer, different polymorphs and solvates can be obtained. The different polymorphs and solvates can be transformed into one-another by crystallization. By using appropriate solvents and applying appropriate physical parameters (reaction conditions) the forms most stable at room temperature, can be obtained.

The synthesis of intermediate (IV) is illustrated by Scheme 2 below:

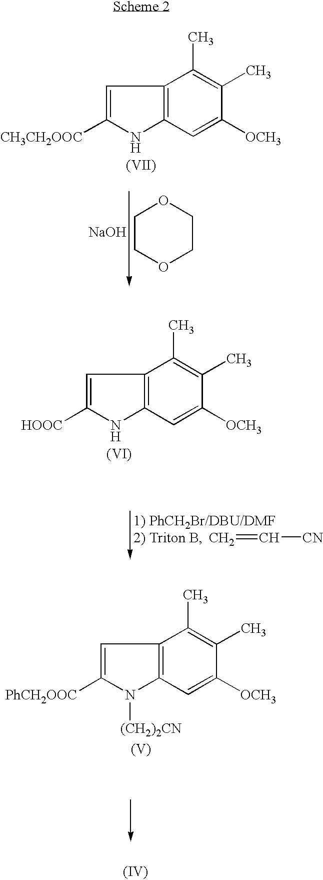

Scheme 3 illustrates the preparation of intermediates (III):

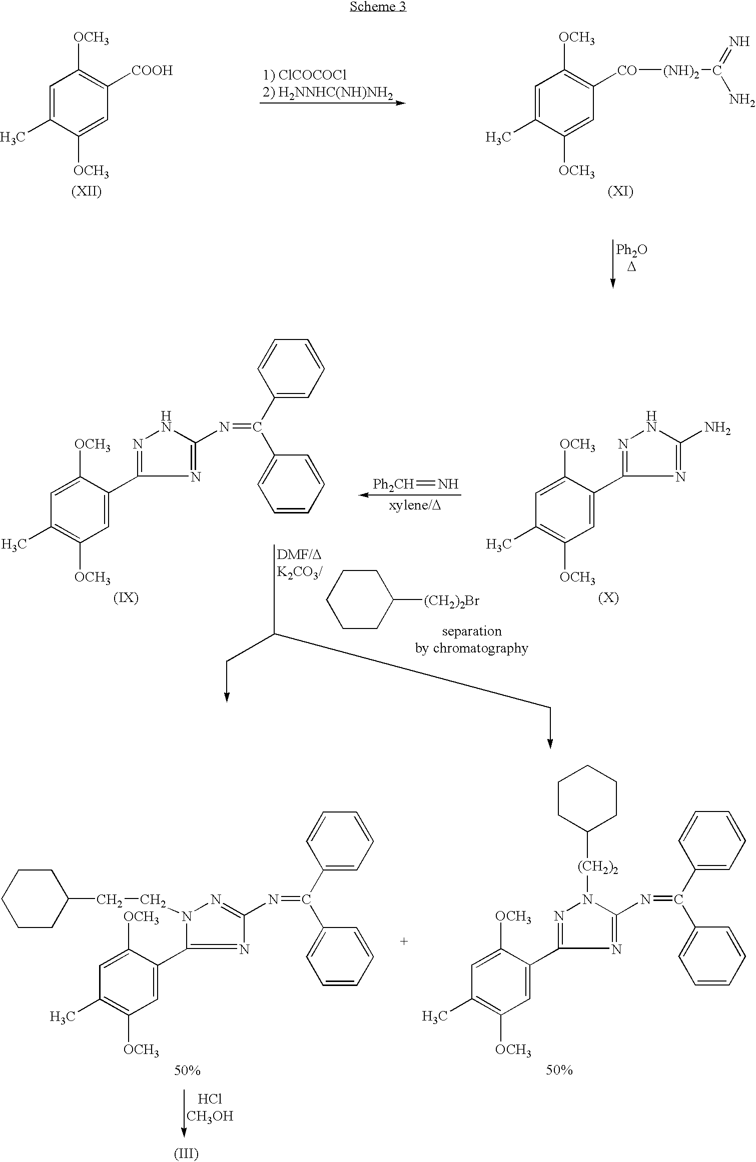

In the above Schemes, the abbreviations Ph for phenyl, DMF for dimethylformamide and DBU for 4,5-dimethyl-6-methoxy-2-indolecarboxylic acid are used.

Polymorphs and solvates of the compounds of the formula (I), their physical characteristics, and conditions of their preparations are presented in Table 1.

TABLE 1
Polymorphs of the acid of the formula (I):

| Code of | | |
|---|---|---|
| the | | |
| poly- | | |
| morph | Preparation conditions | m.p. ° C. |
| (IA) | The sample of the acid of formula (I) is dissolved | 230-231 |
| | in 32-fold (by mass) 96% ethanol at reflux | |
| | temperature, then cooled to 10° C. by a cooling | |
| | rate of 15° C./min., kept at 10° C. for 20 hours, | |
| | filtered off, dried in vacuum oven at 50° C. for 3 | |
| | hours. | |
| (IB) | method a): the sample of polymorph (IA) of the | 230-231 |
| | acid is heated at 160° C. for 6 hours. | |
| | method b): the sample of polymorph (IA) of the | |
| | acid is stirred at a speed of 200 rpm, in silicone | |
| | oil suspension at 180° C. for 6 hours, then cooled | |
| | to room temperature, filtered off after mixing 4 | |
| | times with tert.butyl methyl ether, dried in | |
| | vacuum oven at 50° C. for 1 hour. | |
| | method c): the sample of polymorph (IC) of the | |
| | acid (I) is heated at 200° C. for 6 hours. | |
| | method d): the sample of polymorph (IC) of the | |
| | acid (I) is stirred at a speed of 200 rpm in silicone | |
| | oil suspension, at 200° C. for 6 hours, then cooled | |
| | to room temperature, filtered off after mixing 4 | |
| | times with 1.2-fold (by mass) tert.butyl methyl | |
| | ether, dried in vacuum oven at 50° C. for 1 hour. | |
| (IC) | method a): the sample of the acid of the formula | 211-213; |
| | (I) is dissolved in 30-fold (by mass) 2-propanol at | (melting and |
| | reflux temperature, then cooled to 25° C. at a | crystallizing) |
| | cooling rate of 0.5° C./min., kept at 25° C. for 20 | (229-231)* |
| | hours, filtered off, dried in vacuum oven at 50° C. | |
| | for 3 hours. | |
| | method b): similar result is obtained when the hot | |
| | solution is cooled to 10° C. at a cooling rate of | |
| | 15° C./min., kept at 25° C. for 20 hours, filtered | |
| | off, dried. | |
| | method c): the sample of polymorph (IA) of the | |
| | acid of formula (I) is stirred at a speed of 200 rpm | |
| | in 20-fold (by mass) 96% ethanol at 25-50° C. | |
| | for 3 days, filtered off, dried in vacuum oven at | |
| | 50° C. for 2 hours. | |
| | method d): the sample of polymorph (IA) of the | |
| | acid of formula (I) is stirred at a speed of 200 rpm | |
| | in 25-fold (by mass) n-heptane at 25-90° C. for | |
| | 3-7 days, filtered off, dried in vacuum oven at | |
| | 50° C. for 2 hours. | |
| | method e): similar to method c) but starting from | |
| | polymorph (IE). | |
| | method f): similar to method d) but starting from | |
| | polymorph (IE). | |
| | method g): similar to method c) but starting from | |
| | polymorph (IF). | |
| | method h): the sample of polymorph (IG) of the | |
| | acid of formula (I) is stirred at a speed of 200 rpm | |
| | in 30-fold (by mass) 96% ethanol at 25° C. for 1 | |
| | hour, filtered off, dried in vacuum oven at 50° C. | |
| | for 2 hours. | |
| | method i): the sample of polymorph (IG) of the | |
| | acid of formula (I) is stirred at a speed of 200 rpm | |
| | in 25-fold (by mass) n-heptane at 25° C. for 16 | |
| | days, filtered off, dried in vacuum oven at 50° C. | |
| | for 2 hours. | |
| (ID) | The sample of the acid of formula (I) is dissolved | 222-226 |
| (IDa + | in 40-fold (by mass) 96% ethanol at reflux | |
| IDb) | temperature, then cooled to 25° C. at a cooling | |
| | rate of 0.5° C./min., seeded with the crystals of | |
| | (ID), kept at 25° C. for 20 hours, filtered off, | |
| | dried in vacuum oven at 50° C. for 3 hours. | |
| (IDb) | method a): the sample of polymorph (ID) of the | |
| | acid is stirred at 200 rpm speed in silicone oil | |
| | suspension at 205° C. for 8 hours, then cooled to | |
| | room temperature, filtered off after mixing 4 | |
| | times with 1.5-fold (by mass) tert.butyl methyl | |
| | ether, dried in vacuum oven at 50° C. for 1 hour. | |
| | method b): the sample of polymorph (IC) of the | |
| | acid of formula (I) is stirred at 200 rpm speed in | |
| | 15-fold (by mass) 96% ethanol at 50° C. for 30 | |
| | days, filtered off, dried in vacuum oven at 50° C. | |
| | for 2 hours. | |
| | method C): the sample of polymorph (IC) of the | |
| | acid of formula (I) is stirred at a speed of 200 rpm | |
| | in 15-fold (by mass) 96% ethanol at 70° C. for 12 | |
| | hours, cooled to r.t., filtered off, dried in vacuum | |
| | oven at 50° C. for 2 hours. | |
| | method d): Similar to method c) but starting from | |
| | polymorph (ID). | |
| (IE) | method a): chloroform-solvate pseudopolymorph | 137-140; |
| | (IG) of the acid (I) is dried in vacuum oven at | 168-180 |
| | 80° C. for 3 hours. | (crystal- |
| | method b): The sample of the acid of formula (I) | lization); |
| | is dissolved in 20-fold (by mass) chloroform- | (229-231)* |
| | ethanol 3,75:1 (by mass) mixture, seeded with the | |
| | crystals of (IE), kept at 25° C. for 6 hours, | |
| | filtered off, dried in vacuum oven at 50° C. for | |
| | 3 hours. | |
| (IF) | method a): The sample of the acid of formula (I) | 154-158; |
| | is dissolved in 60-fold (by mass) acetone at reflux | 170-180 |
| | temperature, then cooled to 25° C. at a cooling | (crystal- |
| | rate of 0.5° C./min., kept at 25° C. for 20 hours, | lization); |
| | filtered off, dried in vacuum oven at 50° C. for | (229-231)* |
| | 2 hours. | |
| | method b): the sample of polymorph (IA) of the | |
| | acid of formula (I) is stirred at a speed of 200 rpm | |
| | in 30-fold (by mass) acetone at 25° C. for 8 days, | |
| | filtered off, dried in vacuum oven at 50° C. for 2 | |
| | hours. | |
| | method c): similar to method b) but starting from | |
| | polymorph (IC). | |
| | method d): similar to method b) but starting from | |
| | polymorph (ID). | |
| (IG) | Pseudopolymorph of the acid of the formula (I) | 135-140; |
| | with chloroform, in molar ratio 1:1 | 170-180 |
| | The sample of the acid of formula (I) is dissolved | (crystal- |
| | in 15-fold (by mass) chloroform, kept at 25° C. | lization); |
| | for 1 hour, the precipitate is filtered off and dried | (229-231)* |
| | at room temperature. | |

*Polymorphs (IC), (IE), (IF), (IG) transform into polymorph (IB) by heating above their melting points.

Melting points were determined on a Boetius PHMK 05 type apparatus. Heating rate: 10° C./minute.

The invention also relates to the new salts of the acid of formula (I) and of its polymorphs and solvates, given with

ethanolamine of the formula (A): HO—(CH₂)₂—NH₂, or

diethanolamine of the formula (B): HO—(CH₂)₂—NH—(CH₂)₂—OH, or

diethylamine of the formula (C): (CH₃CH₂)₂NH, or

adamantanamine of the formula (D):

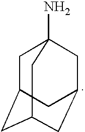

The new salts of the present invention have constant stoichiometry, they are non-hygroscopic, stable, and have favourable technological characteristics for drug product manufacturing. In contrast to the acid of the formula (I), the new salts of the present invention do not show polymorphism, and their solubility in aqueous medium is higher by one order than that of the free acid.

Most favourable properties of the new salts of the present invention are shown by the 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid ethanolamine salt.

The present invention relates further to the process of preparation of the new salts formed between the acid of formula (I), or its polymorphs or solvates, and ethanolamine, diethanolamine, ethylamine, or with adamantanamine, which comprises reacting the acid of formula (I) or a polymorph or solvate of it with

ethanolamine of the formula (A), or

diethanolamine of the formula (B), or

diethylamine of the formula (C), or

adamantanamine of the formula (D).

The compounds of formulae (A), (B), (C) and (D) are preferably applied in a molar excess of 1.0-1.2. Reactions are preferably carried out in a protic solvent, preferably at room temperature. As a protic solvent preferably ethanol, acetone, or ethyl acetate are used.

The compounds of formula (I) underwent studies of in vitro binding to CCK₁ and CCK₂ receptors, using the method described in Europ. J. Pharmacol., 1993, 232, 13-19. Compound of Example 1 binds with a very high affinity (IC₅₀=0.4 nM) (IC₅₀: Inhibiting Concentration₅₀) to the human CCK₁ receptor and with a low affinity to the human CCK₂ receptor (IC₅₀=234 nM), leading to a high level of selectivity (affinity CCK₁ receptor versus affinity of CCK₂ receptor>500-fold). The agonist activity of the compounds towards CCK₁ receptors was evaluated in vitro in 3T3 cells expressing the human CCK₁ receptor, by measuring the mobilization of the intracellular calcium ([Ca⁺⁺]ᵢ), according to a technique derived from that of Lignon M F et al., Eur. J. Pharmacol., 1993, 245, 241-245. The calcium concentration [Ca⁺⁺]ᵢ is evaluated with Fura-2 by the double excitation wavelength method. The ratio of the fluorescence emitted at two wavelengths gives the concentration of [Ca⁺⁺]ᵢ, after calibration (Grynkiewiez G. et al., J. Biol. Chem., 1985, 260, 3440-3450).

The compounds of the invention, like CCK₁ stimulate [Ca⁺⁺]ᵢ release with an efficiency comparable to that of CCK-8S: for compound of Example 1: EC₅₀ (Efficiency Concentration₅₀), around 1 nM and so behave as CCK₁ receptor agonists.

An in vivo study of the agonist effect of the compounds on gastric emptying was carried out as follows. Female Swiss albino CD1 mice (20-25 g) are placed on a solid fast for 18 hours. On the day of the experiment, the products are administered orally 60 minutes before the administration of a charcoal meal (0.3 ml per mouse of a suspension in water of 10% charcoal powder, 5% gum arabic and 1% carboxymethylcellulose). The mice are sacrificed 5 minutes later by cervical dislocation, and the gastric emptying is defined as the presence of charcoal in the intestine beyond the pyloric sphincter (Europ. J. Pharmacol., 1993, 232, 13-19).

The compounds of formula (I) block gastric emptying, like CCK itself, and therefore behave as CCK receptor agonists: compound of Example 3 inhibits gastric emptying at very low doses with an ED₅₀ (Efficient Dose₅₀) of 27 μg/kg p.o.

The compounds of the invention are much more powerful CCK₁ agonists than the molecules described in patent application WO 98/51686. Indeed, surprisingly, they simultaneously meet the following different criteria: they possess not only a high affinity for CCK₁ receptors but also good selectivity for CCK₁ receptors (relative to CCK₂ receptors) and a powerful agonist activity for CCK₁ receptors, demonstrated by the intracellular calcium mobilization and gastric drainage tests.

Consequently, the compounds of formula (I) are used as CCK₁ receptor agonists for preparing medicaments intended for combating diseases whose treatment necessitates stimulation of cholecystokinin CCK₁ receptors. More particularly, the compounds of formula (I) are used for the manufacture of medicaments intended for the treatment of certain disorders of the gastrointestinal field (prevention of bile stones, irritable bowel syndrome, etc), eating disorders, obesity and associated pathologies such as diabetes and hypertension. The compounds (I) induce a state of satiety and are therefore used to regulate appetite and to reduce food intake, to treat obesity and to bring about weight loss. The compounds (I) are also useful in central nervous system disorders, especially disorders of memory loss, sexual disorders and emotional behaviour disorders, psychoses and, in particular, schizophrenia, Parkinson's disease, dyskinesia, such as tardive dyskinesia or facial dyskinesia induced following treatment by neuroleptics or other agents such as dopamine agonists which are used in the treatment of Parkinson's disease, and various disorders of the gastrointestinal field. They may also be used to treat craving disorders, i.e. to regulate the desire to consume—in particular, to consume sugars, fat, alcohol or drugs and, more generally, appetite-inducing ingredients. The compounds (I) are also useful for the treatment and/or prophylaxis of all diseases involving degeneration of NGF-sensitive neurons, such as, for example, cholinergic neurons and sympathic or sensorial neurons, more particularly for the treatment of the following pathologies: memory disorders, vascular dementia, post-encephalitic disorders, post-apoplectic disorders, post-traumatic syndromes due to cranial trauma, disorders deriving from cerebral anoxias, Alzheimer's disease, senile dementia, AIDS-induced dementia, neuropathies as a result of morbidity or damage to sympathic or sensorial nerves, cerebral diseases such as cerebral oedema and spinocerebellar degeneration, and diabetic neuropathies.

The present invention therefore also provides pharmaceutical compositions comprising a compound of the invention together with appropriate excipients.

The said excipients are selected depending on the pharmaceutical form and the desired method of administration: oral, sublingual, subcutaneous, intramuscular, intravenous, topical, intratracheal, intranasal, transdermal, rectal or intraocular. These compositions are prepared in accordance with techniques which are well known to the person skilled in the art.

Each unit dose may contain from 0.1 to 1 000 mg, preferably from 0.1 to 500 mg, of active ingredient in combination with a pharmaceutical excipient.

This unit dose may be administered from 1 to 5 times a day such as to administer a daily dose of from 0.05 to 5 000 mg, preferably from 0.1 to 2 500 mg.

The pharmaceutical compositions of the invention may be used in the treatment or prevention of various conditions in which CCK is of therapeutic interest.

The invention also relates to a method of treatment which comprises using effective doses of a compound of the invention for combating diseases whose treatment necessitates stimulation of cholerystokinin CCK₁ receptors.

The examples below illustrate the invention.

Preparation 1

2,5-Dimethoxy-4-methylbenzoic acid (Compound XII)

a) 2,5-Dimethoxy-4-methylbenzaldehyde

280 ml of phosphorus oxide trichloride are admixed with 212 ml of N-methylformanilide. After 4 hours at room temperature, 110 g of 2,5-dimethoxytoluene are added and the reaction mixture is brought to 70° C. for 2 hours. The reaction mixture is poured dropwise onto ice. The precipitate obtained is filtered, taken up in dichloromethane and decanted. The organic phase is dried over anhydrous sodium sulphate and the solvents are evaporated under reduced pressure. This gives 116 g of yellow crystals; m.p.=83° C.

b) 2,5-Dimethoxy-4-methylbenzoic acid

23.86 g of 2,5-dimethoxy-4-methylbenzaldehyde in solution in 500 ml of water are heated to 75° C. and 29.3 g of potassium permanganate in solution in 500 ml of water are introduced. The reaction mixture is left at 75° C. for 2 hours, after which the pH is adjusted to 10 with 10% sodium hydroxide solution and the insoluble matter is filtered off hot and washed three times with 80 ml of hot water. The filtrate is cooled and the precipitate formed is filtered off and dried under vacuum at 40° C. to give white crystals; m.p.=120° C.; yield=71%

¹H NMR: 2.15 (s, 3H); 3.73 (s, 6H); 6.94 (s, 1H); 7.17 (s, 1H); 12.40 (s, 1H).

Preparation 2

2,5-Dimethoxy-4-methylbenzamidoguanidine (Compound XI)

43.46 g of 2,5-dimethoxy-4-methylbenzoic acid in suspension in 300 ml of toluene are admixed with 1 ml of dimethylformamide and then dropwise with 23.3 ml of oxalyl chloride. The reaction mixture is heated at 80° C. for two hours and then the solvents are evaporated under reduced pressure. The crystalline residue is added in portions to a suspension of 36.2 g of aminoguanidine hydrogen carbonate in 350 ml of pyridine at 0° C. and the reaction mixture is left at ambient temperature for 18 hours. The solvents are evaporated under reduced pressure and then the residue is taken up in 180 ml of water and 141 ml of 2M sodium hydroxide solution. Following 18 hours stirring at ambient temperature, the precipitate is filtered off and dried under reduced pressure to give a beige solid; m.p.=193° C.; yield=93%.

Preparation 3

3-(2,5-Dimethoxy-4-methylphenyl)-1H-1,2,4-triazol-5-amine (Compound X)

29.98 g of 2,5-dimethoxy-4-methylbenzamidoguanidine are admixed with 400 ml of diphenyl ether and then the reaction mixture is heated at 170° C. for 5 minutes. The temperature is taken down to 80° C. and then the precipitate is filtered off, washed with diisopropyl ether and dried under reduced pressure to give crystals; m.p.=248° C.; yield=80%.

Preparation 4

3-(2,5-Dimethoxy-4-methylphenyl)-N-(diphenylmethylene)-1H-1,2,4-triazol-5-amine (Compound IX)

22.4 g of 3-(2,5-dimethoxy-4-methylphenyl)-1H-1,2,4-triazol-5-amine in suspension in 50 ml of xylene and 42 ml of benzophenoneimine are heated at 140° C. for 48 hours under a stream of argon. The temperature is taken down to 80° C. and then the reaction mixture is poured into 100 ml of diisopropyl ether, and the precipitate formed is filtered off, washed with diisopropyl ether and dried under reduced pressure to give a yellow solid; m.p.=228° C.; yield=79%.

Preparation 5

1-(2-Cyclohexylethyl)-5-(2,5-dimethoxy-4-methylphenyl)-1H-1,2,4-triazol-3-amine (Compound III)

a) N-Alkylation of the Triazole

8.8 g of 3-(2,5-dimethoxy-4-methylphenyl)-N-(diphenylmethylene)-1H-1,2,4-triazol-5-amine in solution in 100 ml of dimethylformamide are admixed in successively with 4.5 g of potassium carbonate and 8 ml of 1-bromo-2-cyclohexylethane and the reaction mixture is heated at 70° C. for 18 hours. 300 ml of ethyl acetate are added, the mixture is washed twice with water, the organic phase is dried over anhydrous sodium sulphate and the solvents are evaporated under reduced pressure. The residue is chromatographed on a silica gel column, eluting with a 95/5 (v/v) toluene/ethyl acetate mixture, to give a colourless oil.

¹H NMR: 0.66-1.52 (m, 13H); 2.12 (s, 3H); 3.67 (s, 6H); 3.74 (t, 2H); 6.46 (s, 1H); 6.98 (s, 1H); 7.13-7.71 (m, 10H).

b) Hydrolysis of the Diphenylimine Function

4.7 g of the oil obtained above, in solution in 100 ml of methanol, are admixed with 35 ml of 2M hydrochloric acid. The reaction mixture is left at ambient temperature for 18 hours and then the solvents are evaporated under reduced pressure. The oily residue is concreted in diethyl ether and the precipitate obtained is filtered off and dried under reduced pressure to give white crystals; m.p.=166° C. (HCl); yield=90%.

¹H NMR: 0.82 (m, 2H); 1.05 (m, 4H); 1.3-1.7 (m, 7H); 2.23 (s, 3H); 3.75 (s, 3H); 3.78 (s, 3H); 3.86 (t, 2H); 7.14 (s, 2H); 7.2-7.5 (m, 2H).

Preparation 6

Ethyl 4,5-dimethyl-6-methoxy-1H-indole-2-carboxylate (Compound VII)

Step 1: Preparation of the Azide

2.8 g of sodium are added in portions to 75 ml of ethanol. This solution is admixed dropwise at −20° C. with a mixture of 10 g of 2,3-dimethyl-4-methoxybenzaldehyde and 15.5 g of ethyl azidoacetate in 30 ml of ethanol. After 4 hours at −15° C., the reaction mixture is poured into 400 ml of 1M hydrochloric acid and the precipitate formed is filtered off. It is dried under reduced pressure for 18 hours to give yellow crystals; m.p.=80° C.; yield=65%.

¹H NMR: 1.31 (t, 3H); 2.05 (s, 3H); 2.16 (s, 3H); 3.77 (s, 3H); 4.3 (q, 2H); 6.83 (d, 1H); 7.08 (s, 1H); 7.72 (d, 1H).

Step 2: Cyclization of the Azide

7.9 g of the compound obtained in step 1, in solution in 60 ml of xylene, are added dropwise to 100 ml of xylene heated at 140° C. When the addition is complete, the reaction mixture is left at 140° C. for 5 minutes and returned to ambient temperature. The precipitate obtained is filtered off and dried to give white crystals; m.p.=185° C.; yield=85%.

¹H NMR: 1.3 (t, 3H); 2.1 (s, 3H); 2.35 (s, 3H); 3.76 (s, 3H); 4.27 (q, 2H); 6.69 (s, 1 H); 7.08 (s, 1H); 11.5 (s, 1H).

Preparation 7

4,5-Dimethyl-6-methoxy-1H-indole-2-carboxylic acid (Compound VI)

A mixture of 100 ml of methanol and 150 ml of 1,4-dioxane is admixed with 7 g of ethyl 4,5-dimethyl-6-methoxy-1H-indole-2-carboxylate and then 28 ml of 2M sodium hydroxide solution. The reaction mixture is left at ambient temperature for 48 hours. Following evaporation of the solvents under reduced pressure, the residue is taken up in 6N hydrochloric acid and the precipitate formed is filtered off and dried under reduced pressure to give 4,5-dimethyl-6-methoxy-1H-indole-2-carboxylic acid in the form of white crystals; m.p.=208° C.; yield=92%.

¹H NMR: 2.1 (s, 3H); 2.35 (s, 3H); 3.76 (s, 1H); 6.69 (s, 1H); 7.03 (s, 1H); 11.38 (s, 1H); 12.5 (m, 1H).

Preparation 8

Benzyl 4,5-dimethyl-6-methoxy-1-(2-cyanoethyl)-1H-indole-2-carboxylate (Compound V)

Step 1: Benzyl 4,5-dimethyl-6-methoxy-1H-indole-2-carboxylate

20 ml of dimethylformamide are admixed successively with 5.17 g of 4,5-dimethyl-6-methoxy-1H-indole-2-carboxylic acid and 3.5 ml of 1,8-diazabicyclo[5.4.0]undec-7-ene. The reaction mixture is left at 0° C. for 40 minutes and then 3.9 ml of benzyl bromide are introduced dropwise. After 18 hours of reaction at ambient temperature, the reaction mixture is poured into 300 ml of water and the precipitate formed is filtered off, washed with water and then dried at 50° C. under reduced pressure for 18 hours to give yellow crystals; m.p.=161° C.; yield=90%.

¹H NMR: 2.1 (s, 3H); 2.35 (s, 3H); 3.76 (s, 3H); 5.32 (s, 2H); 6.70 (s, 1H); 7.14 (s, 1H); 7.3-7.55 (m, 5H); 11.57 (s, 1H).

Step 2:

4.24 g of benzyl 4,5-dimethyl-6-methoxy-1H-indole-2-carboxylate in solution in 36 ml of 1,4-dioxane are admixed successively with 0.22 ml of 40% aqueous benzyltrimethylammonium hydroxide solution and 2.18 ml of acrylonitrile and the reaction mixture is heaten to reflux for 4 hours. Following evaporation of the solvents under reduced pressure, the residue is taken up in dichloromethane and washed with water. After decanting, the organic phase is dried over anhydrous sodium sulphate. The residue obtained following evaporation of the organic phase is concreted using diethyl ether and dried to give a beige solid; m.p.=140° C.; yield=95%.

¹H NMR: 2.1 (s, 3H); 2.35 (s, 3H); 2.93 (t, 2H); 3.87 (s, 3H); 4.80 (t, 2H); 5.31 (s, 2H); 7.05 (s, 1H); 7.29-7.50 (m, 6H).

Preparation 9

4,5-Dimethyl-6-methoxy-1-(3-methoxy-3-oxopropyl)-1H-indole-2-carboxylic acid (Compound IV.1)

a) Benzyl 4,5-dimethyl-6-methoxy-1-(3-methoxy-3-oxopropyl)-1H-indole-2-carboxylate

100 ml of methanol are saturated at 0° C. with hydrogen chloride gas. This solution is admixed at −20° C. with 4 g of benzyl 4,5-dimethyl-6-methoxy-1-(2-cyanoethyl)-1H-indole-2-carboxylate in solution in 100 ml of dichloromethane and is left at 0° C. for 18 hours. Following evaporation of the solvents under reduced pressure, the residue is taken up in 60 ml of methanol, 60 ml of dichloromethane and 10 g of ice and is left at 20° C. for 3 hours. The solvents are evaporated and the residue is taken up in ethyl acetate, washed with water and dried over anhydrous sodium sulphate to give a beige solid; m.p.=198° C.; yield=92%.

b) 5.69 g of the compound obtained above are added to 3 g of 10% palladium on carbon in suspension in 500 ml of ethanol. 40 ml of cyclohexene are introduced and the reaction mixture is heaten to reflux for 4 hours. It is filtered at 20° C. and the filtrate is concentrated to give a beige solid; m.p.=198° C.; yield=90%.

### EXAMPLE 1

3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid, Potassium Salt

a) Methyl 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propanoate, compound II.

0.706 g of 4,5-dimethyl-6-methoxy-1-(3-methoxy-3-oxopropyl)-1H-indole-2-carboxylic acid (compound IV) in solution in 5 ml of dichloromethane is admixed successively at 0° C. with 1.08 ml of pyridine and 0.195 ml of thionyl chloride. After 1 hour at this temperature, 0.929 g of 1-(2-cyclohexylethyl)-5-(2,5-dimethoxy-4-methylphenyl)-1H-1,2,4-triazol-3-amine (compound III) is introduced and the reaction mixture is left at 20° C. for 18 hours. Following dilution with dichloromethane and washing with water, the organic phase is dried over anhydrous sodium sulphate and the solvents are evaporated under reduced pressure. The residue is purified by chromatography on a silica gel column, eluting with dichloromethane, to give 1.1 g of white crystals; m.p.=175° C.; yield=83%.

¹H NMR: 0.8 (m, 2H); 1.1 (m, 4H); 1.4-1.7 (m, 7H); 2.12 (s, 3H); 2.23 (s, 3H); 2.37 (s, 3H); 3.55 (s, 3H); 3.74 (s, 6H); 3.84 (s, 3H); 3.9 (t, 2H); 4.37 (t, 2H); 6.89 (s, 1H); 6.91 (s, 1H); 7.06 (s, 1H); 7.52 (s, 1H); 11.54 (s, 1H).

b) 1.59 g of the compound obtained above, in solution in a mixture of 5 ml of methanol and 10 ml of 1,4-dioxane, are admixed with 3 ml of 1 M potassium hydroxide solution and the reaction mixture is left at 20° C. for 72 hours. The solvents are evaporated under reduced pressure and the residue is taken up in diethyl ether, filtered and dried to give 1.56 g of beige crystals; m.p.=236° C.; yield=97%.

¹H NMR: 0.8 (m, 2H); 1.1 (m, 4H); 1.35-1.65 (m, 7H); 2.11 (s, 3H); 2.23 (s, 3H); 2.37 (s, 3H); 2.39 (t, 2H); 3.74 (s, 6H); 3.84 (s, 3H); 3.89 (t, 2H); 4.55 (t, 2H); 6.83 (s, 1H); 6.91 (s, 1H); 7.05 (s, 1H); 7.27 (s 1H); 10.50 (s, 1H).

### EXAMPLE 2

3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid

To a solution of 29.08 g potassium hydroxide in 22.3 ml water and 710 ml ethanol, 95.0 g of the ester of Example 1, step A, is added at 50° C.

After 30 minutes stirring, the mixture is filtered and acidified with 38 ml concentrated HCl in 340 ml water. The precipitate is filtered off, washed with water (to be chloride ion free) and dried to give 90.1 g of the acid; m.p.=222-228° C.; yield: 96.6%.

### EXAMPLE 3

6.17 g of the acid of formula (I) are suspended in 10-fold amount of ethanol and 0.66 g of ethanolamine are added. Clear solution is obtained, allowed to crystallize. The precipitated salt is filtered off, washed with ethanol and dried. 6.2 g of 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid ethanolamine salt are obtained; m.p.=199-200° C.

NMR: 0.79 (m, 2H), 1.06 (m, 4H); 1.4-1.7 (m, 7H); 2.14 (s, 3H); 2.25 (s, 3H); 2.39 (s, 3H); 2.46 (t, 2H, ³J_{CH2,CH2}=7.5 Hz); 2.79 (t, 2H, ³J_{CH2,CH2}=5.2 Hz); 3.55 (t, 2H, ³J_{CH2,CH2}=5.2 Hz); 3.77 (s, 3H); 3.78 (s, 3H); 3.83 (s, 3H); 3.92 (t, 2H, ³J_{CH2,CH2}=7.5 Hz); 4.67 (t, 2H, ³J_{CH2,CH2}=6.9 Hz); 6.90 (s, 1H); 6.94 (s, 1H); 7.08 (s, 1H), 7.48 (s, 1H).

IR: KBr, (cm⁻¹): 3215, 2928, 2846, 2651-2412, 1680, 1622, 1561, 1524, 1485, 1442, 1406, 1262, 1216, 1186, 1144, 1108, 1039, 863, 795, 746.

### EXAMPLE 4

To the solution made of 0.7 g of diethanolamine in 15 ml of ethanol, 3.7 g of the acid (I) are added. The mixture is allowed to stand at room temperature, the resulting crystals are filtered off, washed with ethanol. 3.75 g of 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid diethanolamine salt are obtained; m.p.=171-172° C.

NMR: 0.78 (m,2H); 1.03-1.07 (m, 4H); 1.4-1.7 (m, 7H); 2.13 (s, 3H); 2.23 (s, 3H); 2.38 (s, 3H); 2.46 (t, 2H, ³J_{CH2,CH2}=7.5 Hz); 2.83 (t, 4H, ³J_{CH2,CH2}=5.5 Hz); 3.56 (t, 4H, ³J_{CH2,CH2}=5.5 Hz); 3.74 (s, 3H); 3.76 (s, 3H); 3.84 (s, 3H); 3.91 (t, 2H, ³J_{CH2,CH2}=7.5 Hz); 4.63 (t, 2H, ³J_{CH2,CH2}=7.5 Hz); 6.90 (s, 1H); 6.93 (s, 1H); 7.07 (s, 1H); 7.41 (s, 1H).

IR: KBr, (cm⁻¹): 3439, 2920, 1667, 1620, 1559, 1527, 1478, 1278, 1230, 1146, 1112, 1042, 862, 802, 756, 720.

### EXAMPLE 5

6.2 g of the acid of formula (I) are suspended in 15 ml of ethyl acetate, and 1.5 g of 1-aminoadamantane are added. The resulting clear solution is evaporated. The residue solidifies under hexane to give the 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid adamantanamine salt; m.p.=119° C.

NMR: 0.80 (m, 2H); 1.04-1.08 (m, 4H); 1.4-1.8 (m, 25H); 2.00 (s, 3H); 2.14 (s, 3H); 2.25 (s, 3H); 2.38 (s, 3H); 2.46 (t, 2H, ³J_{CH2,CH2}=7.2 Hz); 3.76 (s, 3H); 3.77 (s, 3H); 3.85 (s, 3H); 3.91 (t, 2H, ³J_{CH2,CH2}=7.2 Hz); 4.65 (t, 2H, ³J_{CH2,CH2}=7.2 Hz); 6.89 (s, 1H); 6.92 (s, 1H); 7.08 (s, 1H); 7.44 (s, 1H); ˜10.8 (b, 1H).

IR: KBr, (cm⁻¹): 3425, 2921, 2851, 1677, 1619, 1560, 1489, 1391, 1217, 1144, 1123, 1042, 863, 801, 757.

### EXAMPLE 6

To the suspension of 3.07 g of the acid of formula (I) in acetone, 0.45 g of diethylamine in 11 ml acetonic of solution are added. The clear solution is concentrated, diethyl ether is added, the resulting crystals are filtered off to obtain:

3.2 g of 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid diethylamine salt; m.p.=143° C. (decomposition).

NMR: 0.79 (m, 2H); 1.03-1.2 (m, 10H); 1.4-1.7 (m, 7H); 2.15 (s, 3H); 2.52 (s, 3H); 2.39 (s, 3H); 2.49 (m, 2H); 2.75 (q, 4H); 3.76 (s, 3H); 3.78 (s, 3H); 3.85 (s, 3H); 3.92 (t, 2H, ³J_{CH2,CH2}=7.1 Hz); 4.67 (t, 2H, ³J_{CH2,CH2}=7.1 Hz); 6.91 (s, 1H); 6.93 (s, 1H); 7.09 (s, 1H); 7.48 (s, 1H); ˜10.8 (b, 1H).

IR: KBr, (cm⁻¹): 3419, 2924, 2850, 1675, 1620, 1555, 1519, 1487, 1390, 1217, 1144, 1112, 1043, 867, 803, 757.

### EXAMPLE7

6.3 g of the methyl ester of the acid of formula (I) are dissolved in 50 ml of 96% ethanol which contains 2 g of potassium hydroxide. The solution is kept at 45-50° C. for 40 minutes. After clarifying with charcoal and filtration, the pH is adjusted to 3 with aqueous hydrochloric acid. The resulting crystals are filtered off, washed thoroughly with water. 5.9 g of the acid of formula (I) are obtained. Purity by HPLC: 98.9%; m.p.=234° C.

### EXAMPLE 8

6.03 g of the methyl ester of the acid of formula (I) are dissolved in 60 ml of 96% ethanol which contains 1.2 g of sodium hydroxide. The solution is stirred at 50° C. for 1 hour, clarified with charcoal, filtered, the warm solution is made acidic, allowed to cool down. 6.03 g of the acid of formula (I) are obtained. Purity by HPLC: 99%. m.p.=213° C. (shrinking) −231° C. (melting).

### EXAMPLE 9

The following solid forms of the compound of formula (I) have been identified, by using the methods of investigation shown below:

Polymorphs:

polymorph (IA)

polymorph (IB)

polymorph (IC)

polymorph (IDb)

polymorph (IE)

polymorph (IF)

Solvates:

Solvate (pseudopolymorphs) (IG), which is the solvate of polymorph (IE) with CHCl₃.

Mixture form: polymorph (ID), which is most likely the mixture of polymorph (IDb) with another polymorph which has not been obtained in pure state, as yet.

Methods of investigation

Conditions

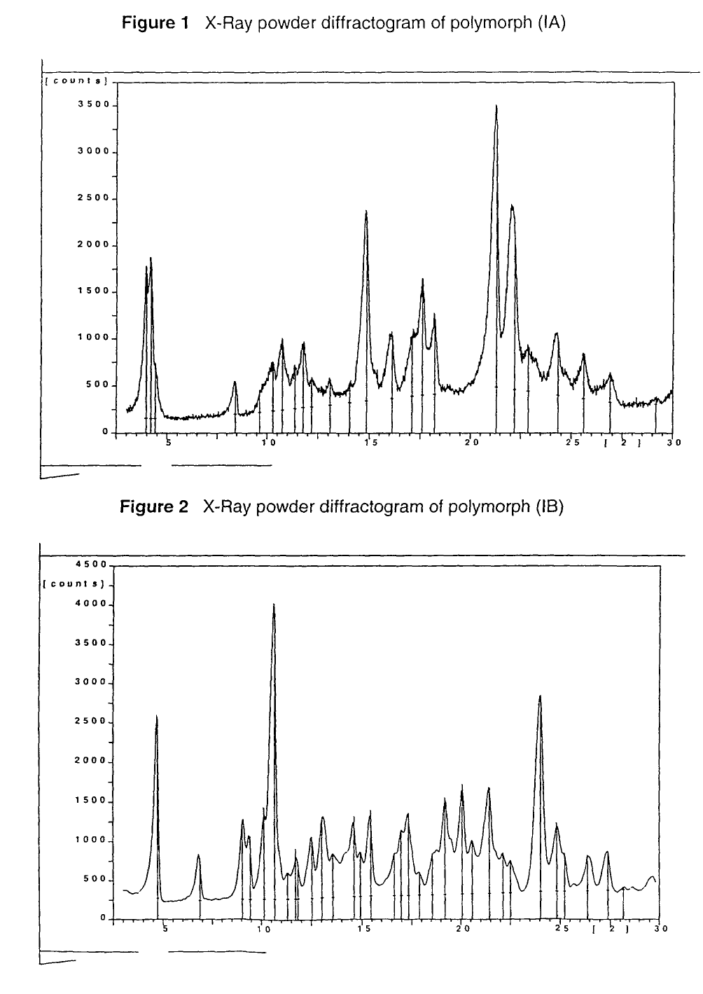

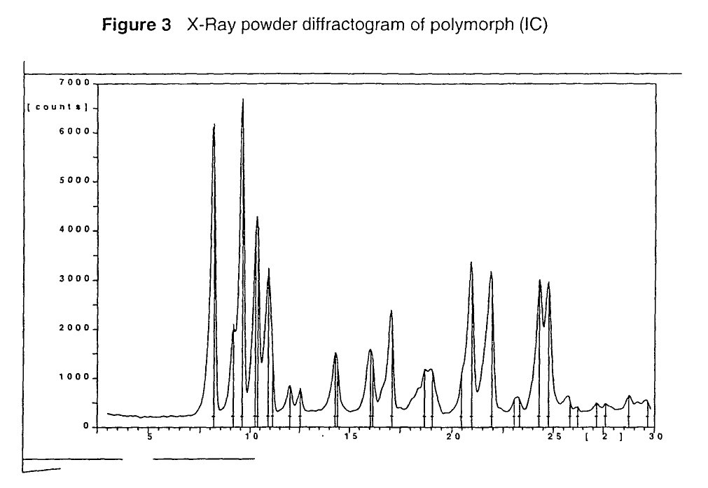

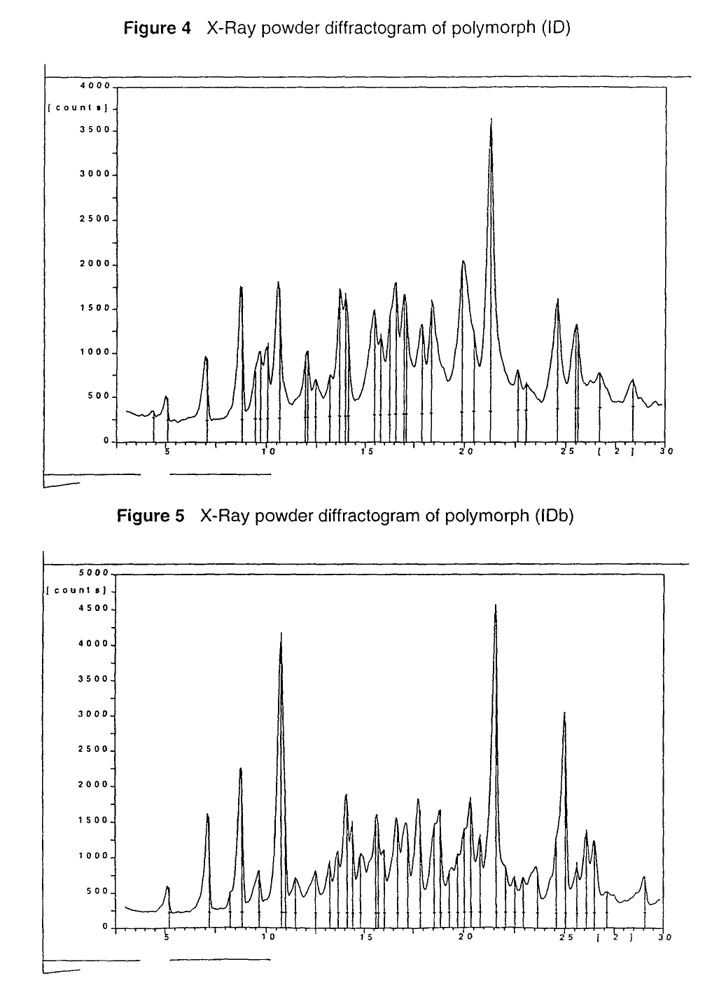

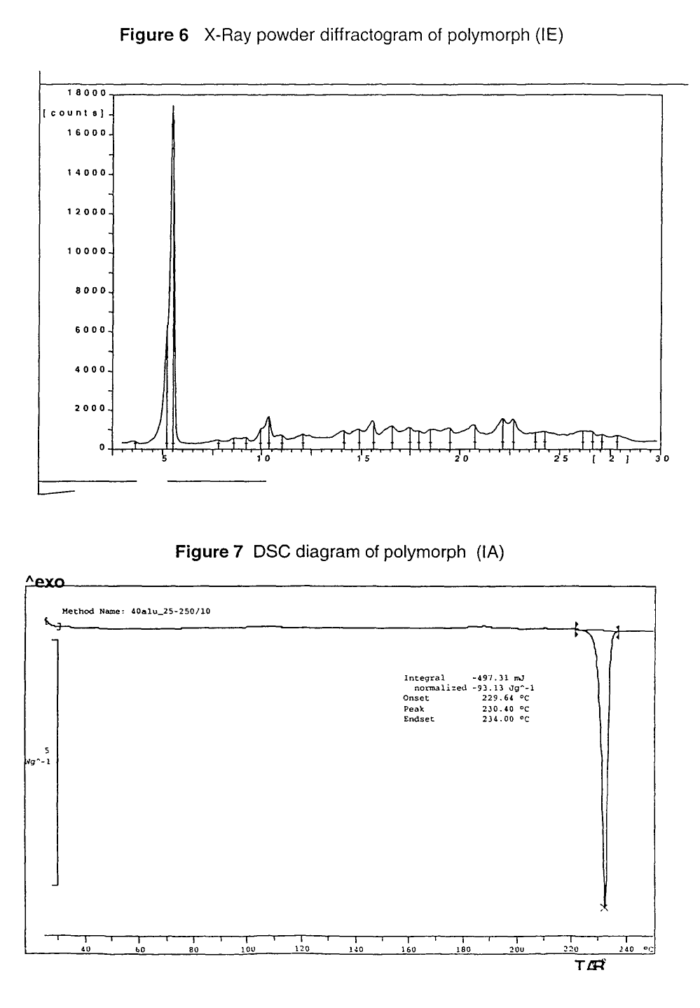

X-RAY POWDER DIFFRACTION

| | |
|---|---|
| Instrument | Philips powder diffractometer |
| | PW3710 |
| Radiation | CuKα (λ = 1.5418 Å |
| Lambda α1 (Å) | 1.54060 |
| Lambda α2 (Å) | 1.54439 |
| α1:α2 ratio | 2:1 |
| 2Θ Range | 3-30° |
| Scanning speed (2Θ°) | 0.02 |
| Scanning interval (mp) | 1 |
| see | Table 3 and FIGS. 1-6 |

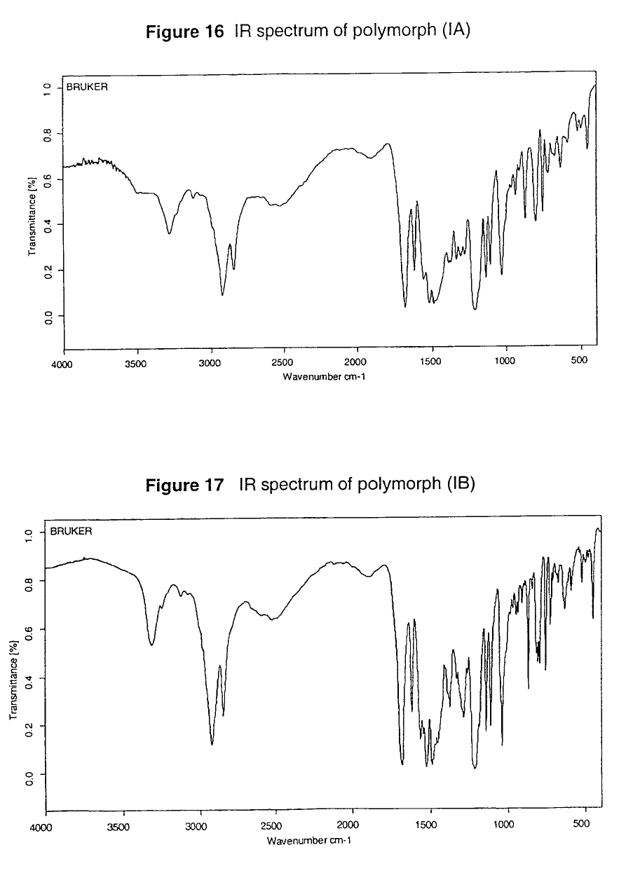

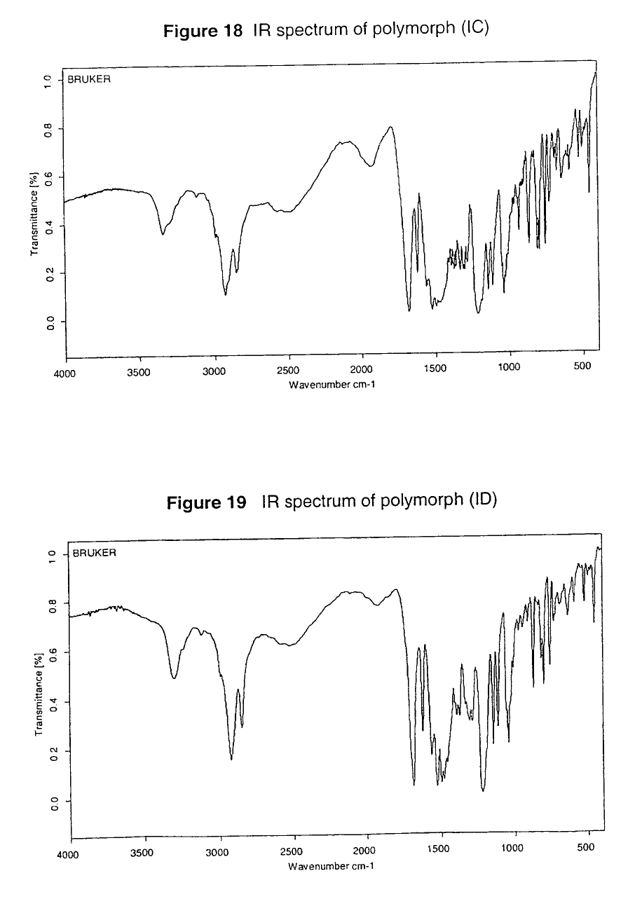

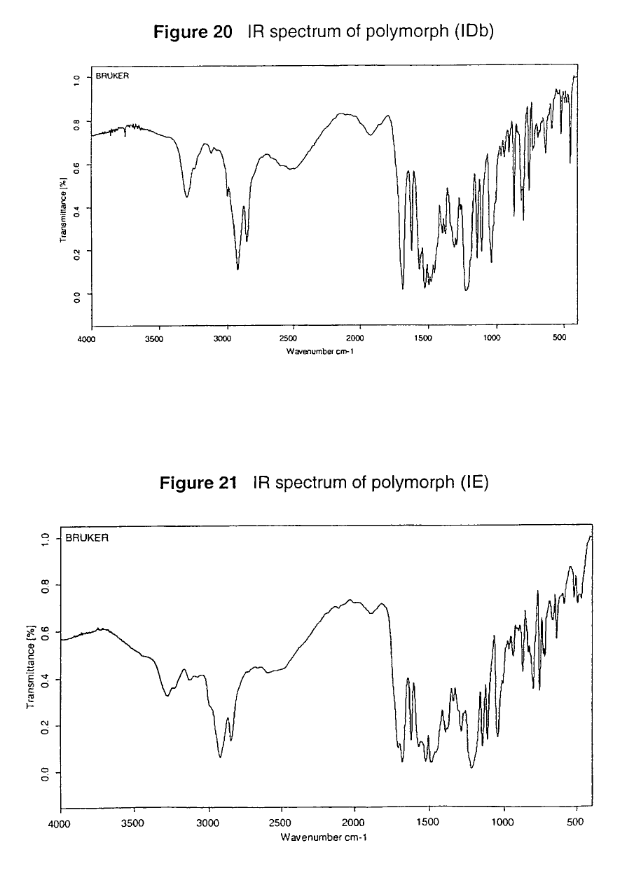

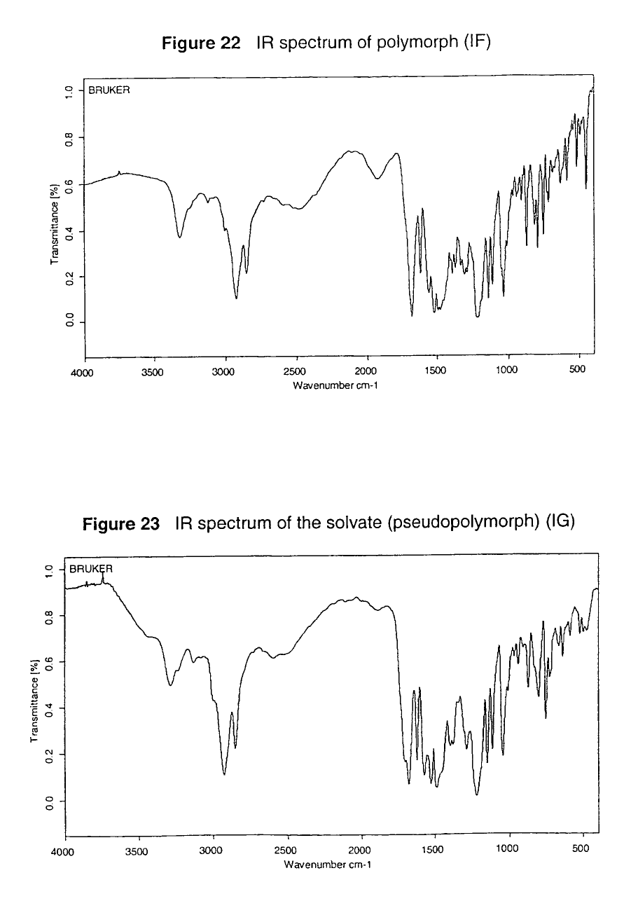

IR SPECTROSCOPY

| | |
|---|---|
| Instrument | Bruker IFS-28 |
| Range | 4000-400 cm⁻¹ |
| Sample preparation | 1-2 mg of sample 0.2 g of KBr |
| | compressed in pellett |
| See | Table 2 and FIGS. 16-23 |

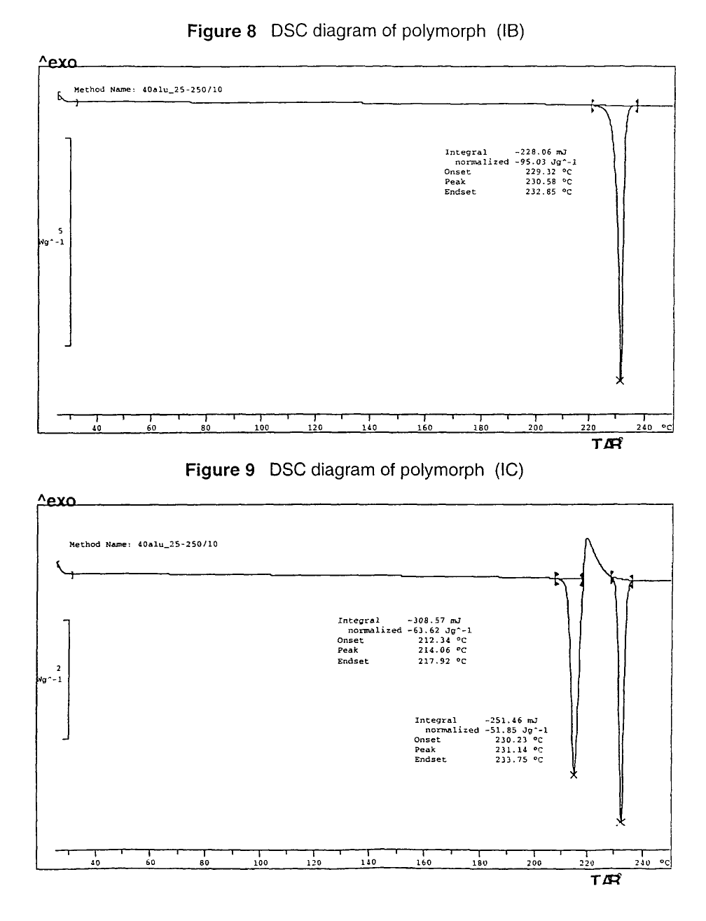

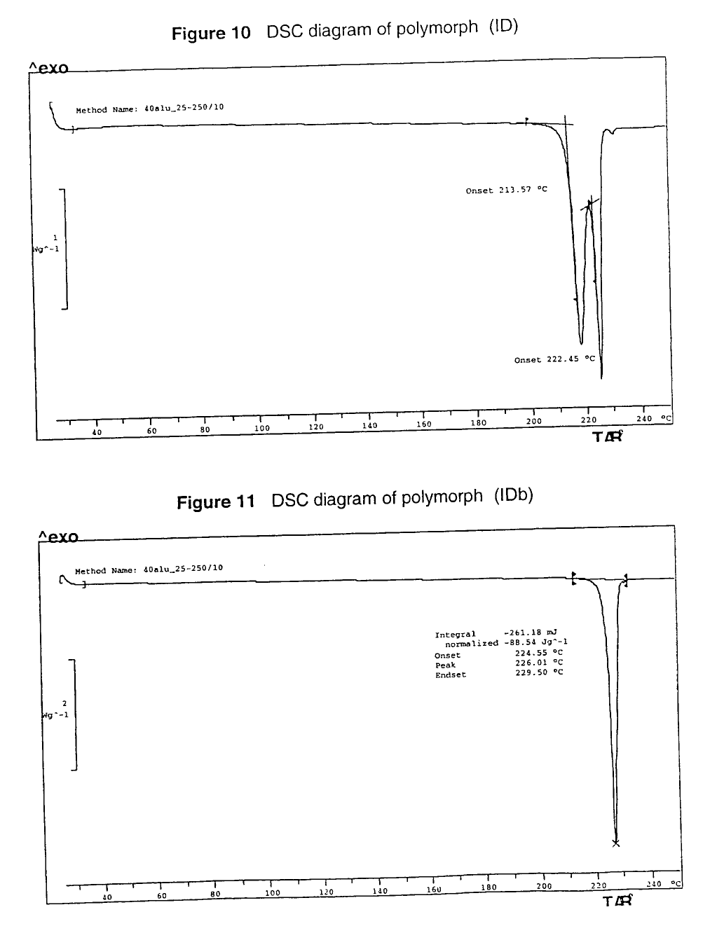

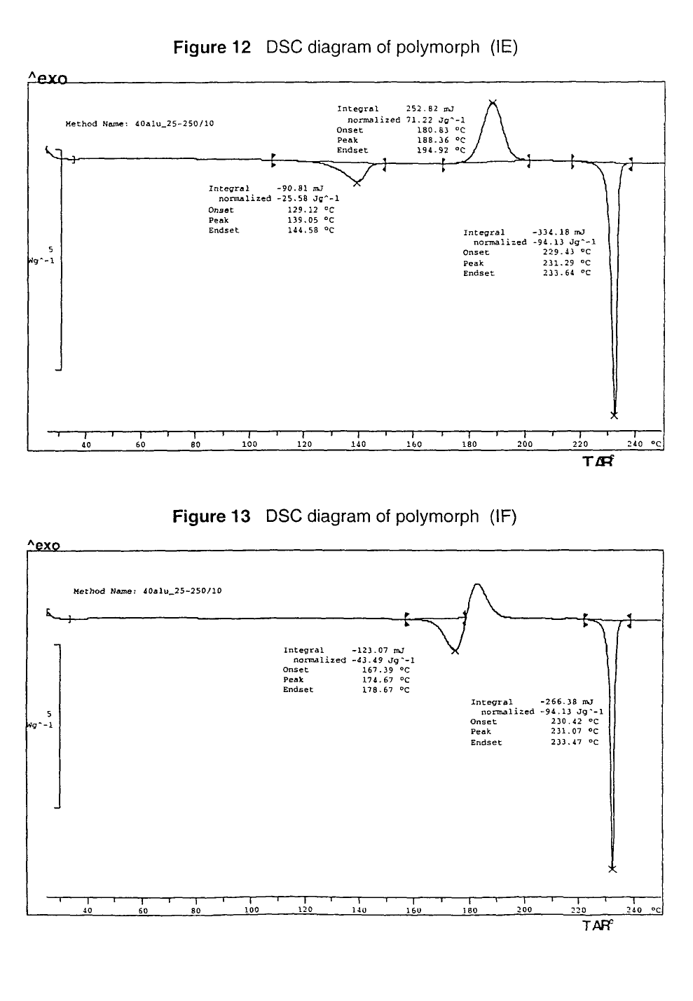

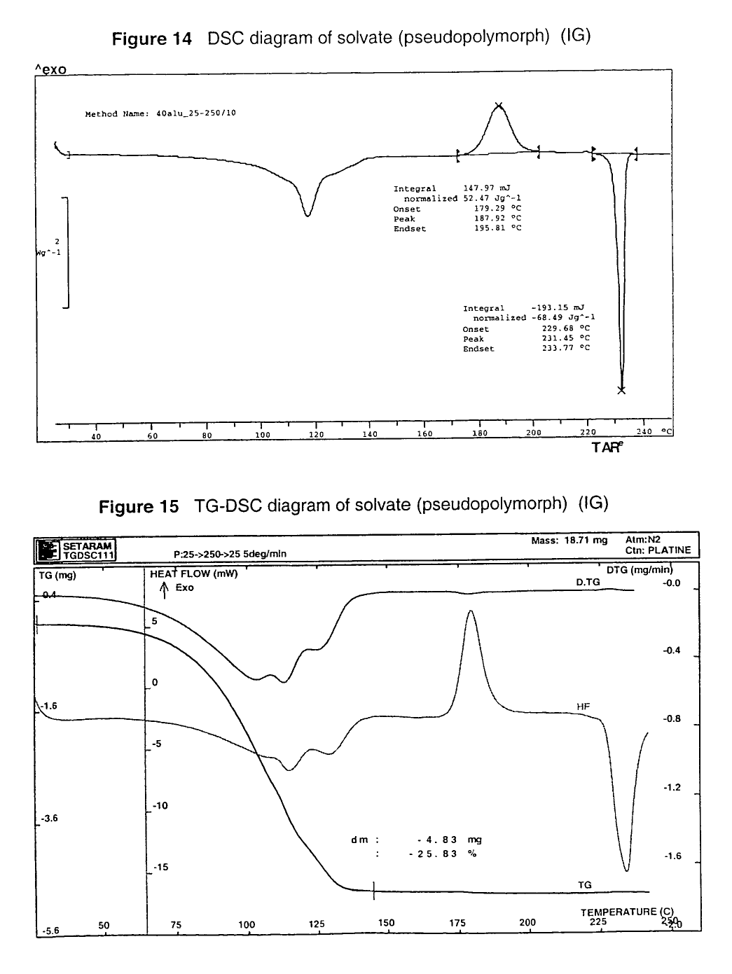

DSC

| | |
|---|---|
| Instrument | Mettier Toledo DSC821e |
| Temperature range | 25-250° C. |
| Heating rate | 10° C./minute |
| Sample holder | 40 μl alumina crucible, cover |
| | with hole |
| Gas flow | Air, 0 ml/perc |
| See | Table 5 and FIGS. 7-14 |

TG-DSC

| | |
|---|---|
| Instrument | Setaram TG-DSC111 |
| | simultanous TG-DSC |
| | measurements |
| Temperature range | 25-250° C. |
| Heating rate | 5° C./minutes |
| Sample holder | Platinum crucible |
| Gas flow | N₂ |
| See | Tables 5 and 15. |

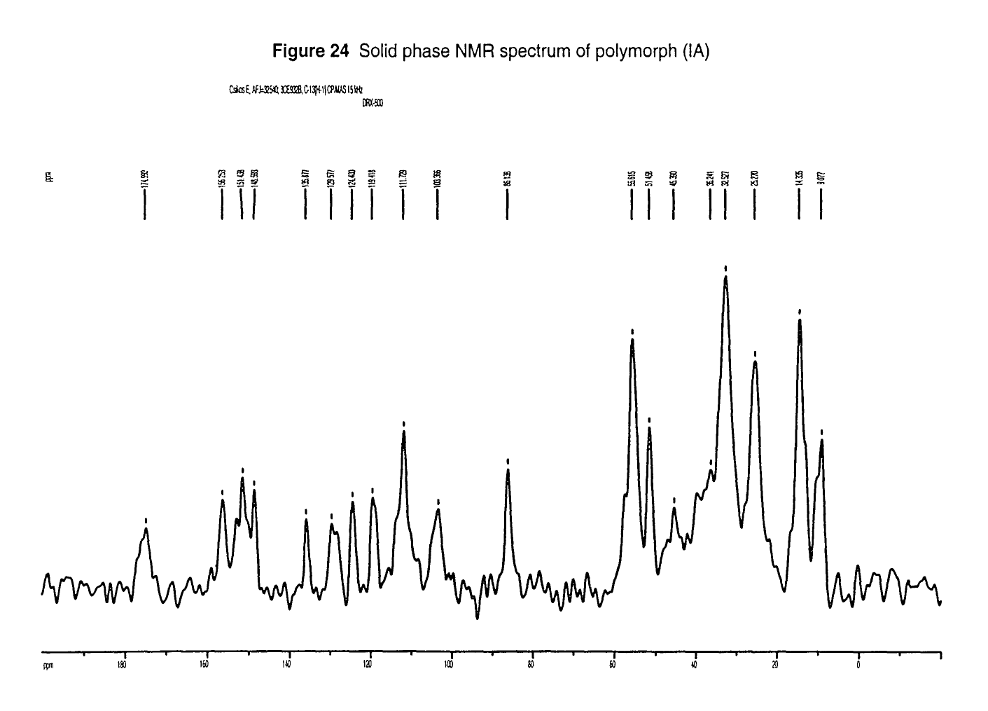

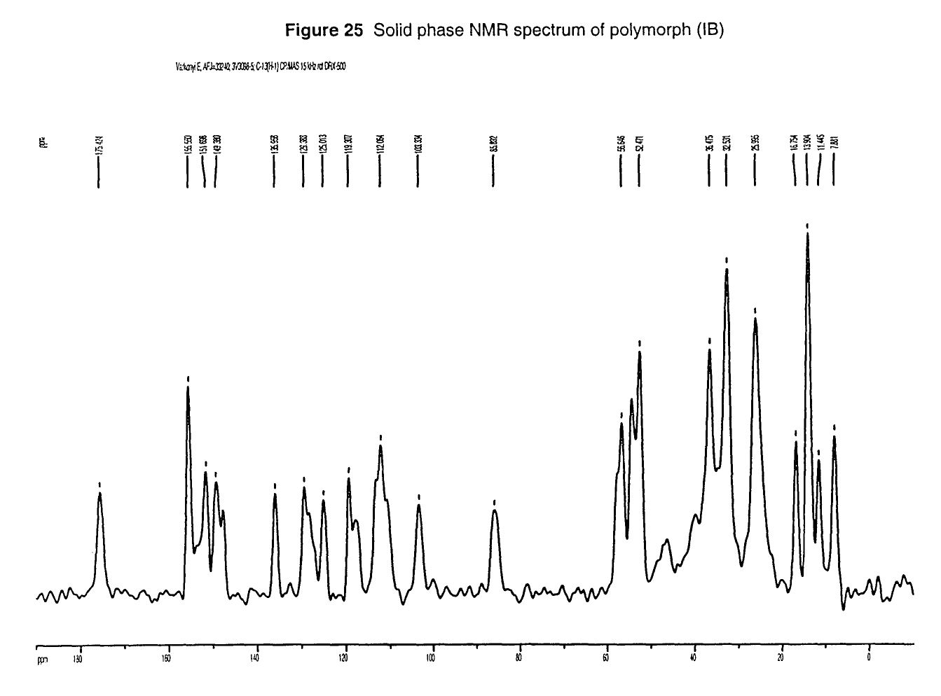

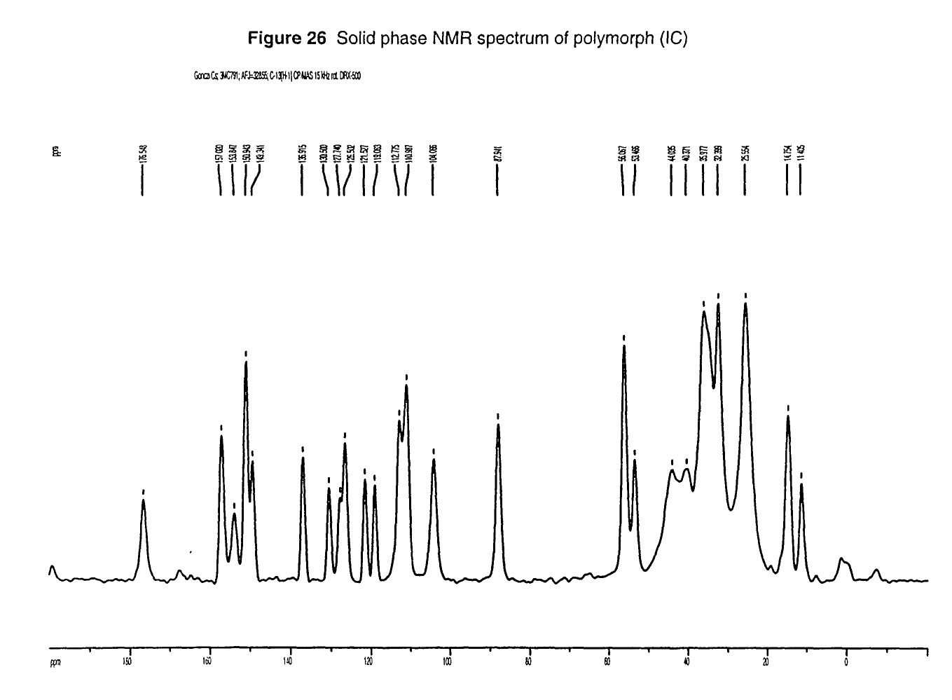

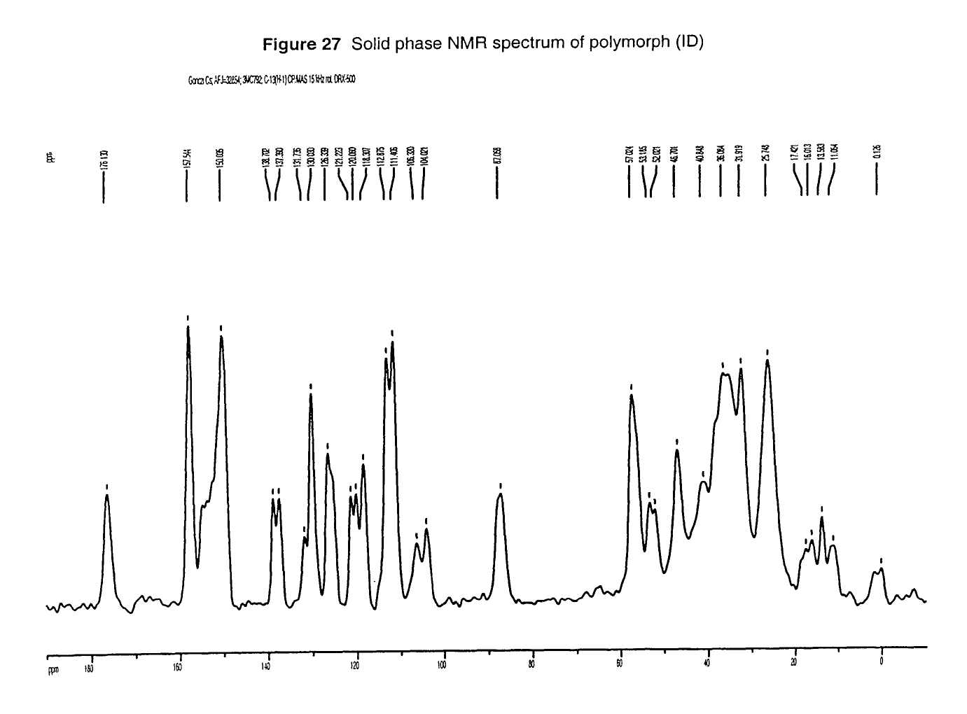

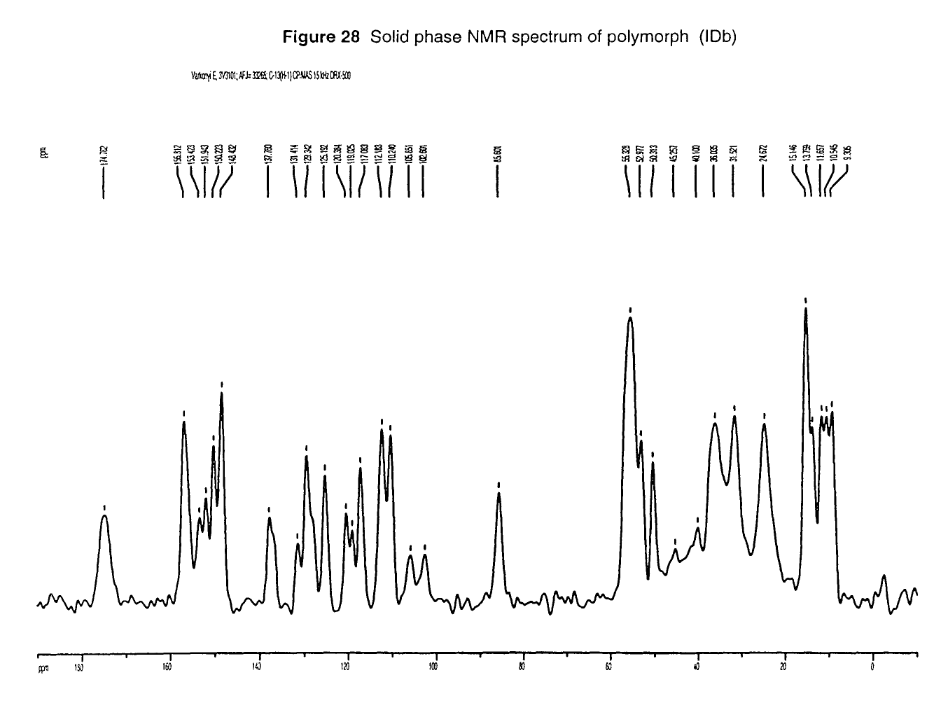

SOLID PHASE NMR

| | |
|---|---|
| Instrument | Bruker DRX-500 |
| Measurement | ¹³C (¹H) CP/MAS |
| Spinning rate | 15 KHz |
| See | Table 4 and FIGS. 24-28 |

TABLE 2
IR spectroscopic characteristics

| Polymorph (IA) | | Polymorph (IB) | | Polymorph (IC) | | Polymorph (ID) | |
|---|---|---|---|---|---|---|---|
| | Rel. | | Rel. | | Rel. | | Rel. |
| Wave | Inten- | Wave | Inten- | Wave | Inten- | Wave | Inten- |
| number | sity | number | sity | number | sity | number | sity |
| (cm⁻¹) | (I/Iₒ) | (cm⁻¹) | (I/Iₒ) | (cm⁻¹) | (I/Iₒ) | (cm⁻¹) | (I/Iₒ) |
| 3281.3 | 0.221 | 3309.7 | 0.279 | 3337.8 | 0.186 | 3299.1 | 0.232 |
| 3118.8 | 0.032 | 3121.6 | 0.030 | 2926.0 | 0.540 | 3116.5 | 0.024 |
| 2925.1 | 0.605 | 2921.0 | 0.760 | 2851.8 | 0.108 | 2920.9 | 0.647 |
| 2847.7 | 0.121 | 2848.7 | 0.223 | 2516.5 | 0.082 | 2849.0 | 0.164 |
| 2523.8 | 0.084 | 2524.1 | 0.126 | 1935.6 | 0.132 | 2525.3 | 0.082 |
| 1905.6 | 0.056 | 1897.4 | 0.054 | 1681.9 | 0.612 | 1921.6 | 0.061 |
| 1684.9 | 0.577 | 1683.7 | 0.611 | 1620.8 | 0.297 | 1683.4 | 0.671 |
| 1619.4 | 0.288 | 1620.1 | 0.378 | 1560.0 | 0.126 | 1618.5 | 0.343 |
| 1559.0 | 0.127 | 1564.6 | 0.170 | 1522.2 | 0.472 | 1559.6 | 0.200 |
| 1520.3 | 0.123 | 1545.5 | 0.037 | 1493.6 | 0.052 | 1522.9 | 0.609 |
| 1490.1 | 0.425 | 1525.9 | 0.513 | 1406.6 | 0.036 | 1493.5 | 0.160 |
| 1386.5 | 0.060 | 1490.3 | 0.217 | 1391.4 | 0.067 | 1476.1 | 0.058 |
| 1336.0 | 0.133 | 1453.2 | 0.042 | 1375.8 | 0.102 | 1390.8 | 0.059 |
| 1308.1 | 0.070 | 1375.1 | 0.216 | 1363.1 | 0.079 | 1371.5 | 0.178 |
| 1282.7 | 0.072 | 1329.4 | 0.044 | 1335.7 | 0.152 | 1305.0 | 0.070 |
| 1215.9 | 0.838 | 1287.8 | 0.258 | 1303.5 | 0.154 | 1286.9 | 0.220 |
| 1143.4 | 0.266 | 1217.3 | 0.960 | 1286.2 | 0.112 | 1218.2 | 0.892 |
| 1111.2 | 0.260 | 1145.2 | 0.449 | 1218.5 | 0.855 | 1142.4 | 0.369 |
| 1033.6 | 0.480 | 1113.8 | 0.420 | 1143.8 | 0.220 | 1109.6 | 0.340 |
| 934.3 | 0.117 | 1038.3 | 0.658 | 1113.8 | 0.221 | 1036.9 | 0.539 |
| 908.0 | 0.046 | 963.9 | 0.043 | 1036.4 | 0.448 | 1004.8 | 0.037 |
| 869.4 | 0.299 | 942.3 | 0.093 | 963.7 | 0.043 | 964.6 | 0.053 |
| 801.4 | 0.347 | 930.0 | 0.055 | 929.7 | 0.226 | 938.2 | 0.059 |
| 754.6 | 0.369 | 904.8 | 0.079 | 899.6 | 0.045 | 904.9 | 0.076 |
| 720.6 | 0.156 | 863.7 | 0.456 | 862.8 | 0.380 | 866.7 | 0.374 |
| 676.5 | 0.071 | 838.2 | 0.059 | 832.0 | 0.026 | 813.5 | 0.107 |
| 637.1 | 0.152 | 813.9 | 0.089 | 807.4 | 0.201 | 797.3 | 0.380 |
| 588.4 | 0.071 | 805.3 | 0.133 | 794.8 | 0.429 | 755.8 | 0.363 |
| 521.7 | 0.078 | 793.5 | 0.416 | 753.8 | 0.460 | 729.6 | 0.165 |
| 499.2 | 0.047 | 756.3 | 0.475 | 726.7 | 0.281 | 687.7 | 0.057 |
| 456.5 | 0.236 | 725.9 | 0.281 | 688.8 | 0.078 | 632.2 | 0.143 |
| | | 708.6 | 0.045 | 672.3 | 0.166 | 588.2 | 0.115 |
| | | 675.0 | 0.079 | 641.3 | 0.216 | 520.0 | 0.156 |
| | | 632.8 | 0.217 | 602.3 | 0.036 | 494.4 | 0.046 |
| | | 590.5 | 0.106 | 589.0 | 0.123 | 453.8 | 0.294 |
| | | 520.9 | 0.146 | 523.9 | 0.196 | | |
| | | 497.5 | 0.039 | 500.8 | 0.145 | | |
| | | 480.4 | 0.033 | 454.1 | 0.431 | | |
| | | 453.1 | 0.355 | | | | |
| | | | | | | Solvate | |
| | | | | | | (Pseudo- | |
| Polymorph (IDb) | | Polymorph (IE) | | Polymorph (IF) | | polymorph) (IG) | |
| | Rel. | | Rel. | | Rel. | | Rel. |
| Wave | inten- | Wave | inten- | Wave | inten- | Wave | inten- |
| number | sity | number | sity | number | sity | number | sity |
| (cm⁻¹) | (I/Iₒ) | (cm⁻¹) | (I/Iₒ) | (cm⁻¹) | (I/Iₒ) | (cm⁻¹) | (I/Iₒ) |
| 3296.9 | 0.270 | 3276.5 | 0.150 | 3316.4 | 0.218 | 3282.7 | 0.216 |
| 3116.5 | 0.034 | 3133.5 | 0.038 | 3116.5 | 0.036 | 3125.9 | 0.048 |
| 2995.0 | 0.056 | 2923.0 | 0.592 | 2921.6 | 0.588 | 2923.2 | 0.774 |
| 2920.6 | 0.695 | 2849.0 | 0.115 | 2850.7 | 0.135 | 2849.0 | 0.171 |
| 2851.4 | 0.178 | 2593.6 | 0.051 | 2484.4 | 0.126 | 2596.5 | 0.077 |
| 2524.2 | 0.129 | 1889.8 | 0.048 | 1924.6 | 0.113 | 1891.4 | 0.031 |
| 1922.0 | 0.096 | 1678.8 | 0.394 | 1683.8 | 0.512 | 1678.1 | 0.431 |
| 1683.7 | 0.593 | 1619.1 | 0.255 | 1619.9 | 0.267 | 1619.4 | 0.307 |
| 1618.0 | 0.369 | 1568.5 | 0.072 | 1560.1 | 0.155 | 1569.6 | 0.200 |
| 1561.8 | 0.223 | 1523.7 | 0.149 | 1522.6 | 0.417 | 1523.8 | 0.205 |
| 1524.4 | 0.523 | 1479.6 | 0.402 | 1493.8 | 0.090 | 1480.7 | 0.419 |
| 1494.1 | 0.124 | 1387.8 | 0.121 | 1392.2 | 0.118 | 1390.7 | 0.132 |
| 1476.3 | 0.053 | 1336.2 | 0.057 | 1371.7 | 0.089 | 1374.5 | 0.044 |
| 1451.7 | 0.064 | 1283.4 | 0.127 | 1331.9 | 0.055 | 1283.1 | 0.136 |
| 1391.6 | 0.084 | 1216.5 | 0.855 | 1304.5 | 0.149 | 1216.4 | 0.886 |
| 1369.4 | 0.191 | 1145.4 | 0.245 | 1286.5 | 0.060 | 1146.8 | 0.281 |
| 1305.0 | 0.250 | 1110.7 | 0.245 | 1217.2 | 0.860 | 1110.9 | 0.308 |
| 1287.6 | 0.087 | 1040.7 | 0.445 | 1142.3 | 0.275 | 1040.9 | 0.485 |
| 1260.4 | 0.039 | 964.2 | 0.043 | 1111.5 | 0.250 | 1005.6 | 0.029 |
| 1227.9 | 0.912 | 935.5 | 0.110 | 1034.5 | 0.479 | 964.7 | 0.044 |
| 1141.9 | 0.409 | 898.8 | 0.021 | 1006.5 | 0.035 | 936.7 | 0.103 |
| 1109.6 | 0.364 | 870.0 | 0.242 | 964.5 | 0.044 | 870.3 | 0.233 |
| 1037.2 | 0.521 | 833.4 | 0.066 | 939.0 | 0.082 | 801.1 | 0.309 |
| 964.4 | 0.057 | 799.8 | 0.384 | 905.0 | 0.114 | 756.7 | 0.428 |
| 939.7 | 0.093 | 757.0 | 0.431 | 870.0 | 0.345 | 731.0 | 0.091 |
| 904.6 | 0.116 | 731.5 | 0.065 | 815.1 | 0.145 | 665.4 | 0.082 |
| 866.9 | 0.433 | 720.8 | 0.185 | 794.6 | 0.393 | 640.0 | 0.139 |
| 813.5 | 0.113 | 667.8 | 0.101 | 755.9 | 0.316 | 588.9 | 0.069 |
| 797.8 | 0.498 | 640.8 | 0.201 | 721.2 | 0.219 | 520.5 | 0.135 |
| 756.5 | 0.394 | 590.3 | 0.061 | 692.2 | 0.061 | 496.3 | 0.077 |
| 728.9 | 0.214 | 521.6 | 0.104 | 636.1 | 0.162 | 473.1 | 0.048 |
| 689.8 | 0.071 | 496.8 | 0.207 | 589.5 | 0.196 | | |
| 633.6 | 0.269 | 471.8 | 0.046 | 543.7 | 0.051 | | |
| 588.3 | 0.133 | | | 517.8 | 0.226 | | |
| 538.0 | 0.022 | | | 493.8 | 0.073 | | |
| 520.2 | 0.201 | | | 452.2 | 0.396 | | |
| 494.9 | 0.052 | | | | | | |
| 478.7 | 0.046 | | | | | | |
| 452.2 | 0.392 | | | | | | |

TABLE 3
X-Ray powder diffractometry Data

| Polymorph (IA) | | Polymorph (IB) | | Polymorph (IC) | | Polymorph (ID) | | Polymorph (IDb) | | Polymorph (IE) | |
|---|---|---|---|---|---|---|---|---|---|---|---|
| 2Θ (°) | I/Iₒ* | 2Θ (°) | I/Iₒ | 2Θ (°) | I/Iₒ | 2Θ (°) | I/I_{o*} | 2Θ (°) | I/Iₒ | 2Θ (°) | I/Iₒ |
| 4.0 | 58 | 4.725 | 63 | 8.2 | 92 | 4.4 | 2 | 5.2 | 8 | 3.7 | 1 |
| 4.2 | 60 | 6.87 | 15 | 9.1 | 29 | 5.1 | 7 | 7.2 | 32 | 5.2 | 35 |
| 4.4 | 21 | 9.035 | 28 | 9.6 | 100 | 7.0 | 21 | 8.2 | 7 | 5.5 | 100 |
| 8.4 | 12 | 9.435 | 22 | 10.3 | 56 | 8.8 | 45 | 8.8 | 48 | 7.8 | 1 |
| 9.7 | 8 | 10.13 | 32 | 10.4 | 59 | 9.4 | 16 | 9.6 | 14 | 8.6 | 2 |
| 10.3 | 17 | 10.66 | 100 | 10.9 | 47 | 9.7 | 23 | 10.7 | 95 | 9.2 | 2 |
| 10.7 | 25 | 11.31 | 9 | 11.1 | 20 | 10.0 | 26 | 10.9 | 41 | 10.0 | 4 |
| 11.4 | 16 | 11.71 | 17 | 12.0 | 9 | 10.6 | 45 | 11.4 | 12 | 10.4 | 8 |
| 11.8 | 24 | 11.835 | 12 | 12.5 | 9 | 11.9 | 21 | 12.5 | 14 | 11.0 | 2 |
| 12.2 | 10 | 12.525 | 21 | 14.3 | 20 | 12.0 | 23 | 13.2 | 18 | 12.1 | 3 |
| 13.1 | 9 | 13.02 | 28 | 14.4 | 17 | 12.4 | 13 | 13.6 | 21 | 14.1 | 4 |
| 14.0 | 8 | 13.55 | 15 | 16.1 | 21 | 13.2 | 14 | 14.1 | 40 | 14.9 | 4 |
| 14.9 | 69 | 14.61 | 28 | 16.2 | 18 | 13.7 | 44 | 14.4 | 31 | 15.6 | 6 |
| 16.1 | 21 | 14.92 | 15 | 17.1 | 31 | 13.9 | 42 | 14.8 | 20 | 16.6 | 5 |
| 17.1 | 23 | 15.445 | 30 | 18.7 | 15 | 14.1 | 28 | 15.6 | 31 | 17.5 | 5 |
| 17.6 | 45 | 16.63 | 14 | 19.1 | 14 | 15.4 | 36 | 15.7 | 31 | 17.9 | 4 |
| 18.2 | 28 | 16.97 | 23 | 20.5 | 13 | 15.8 | 28 | 16.0 | 21 | 18.5 | 4 |
| 21.3 | 100 | 17.335 | 29 | 21.0 | 45 | 16.2 | 33 | 16.7 | 31 | 19.5 | 4 |
| 22.2 | 59 | 17.895 | 8 | 21.9 | 43 | 16.5 | 45 | 17.2 | 27 | 20.8 | 5 |
| 22.9 | 16 | 18.56 | 14 | 23.1 | 5 | 17.0 | 41 | 17.8 | 35 | 22.2 | 7 |
| 24.3 | 21 | 19.2 | 34 | 23.4 | 5 | 17.1 | 36 | 18.5 | 29 | 22.7 | 7 |
| 25.6 | 17 | 20.07 | 38 | 24.3 | 43 | 17.9 | 30 | 18.8 | 35 | 23.8 | 3 |
| 26.9 | 11 | 20.57 | 18 | 24.8 | 42 | 18.3 | 39 | 19.3 | 14 | 24.3 | 3 |
| 29.2 | 2 | 21.45 | 37 | 25.8 | 5 | 19.9 | 52 | 19.7 | 20 | 26.2 | 3 |
| | | 22.13 | 14 | 26.2 | 3 | 20.4 | 27 | 20.0 | 28 | 26.6 | 3 |
| | | 22.505 | 11 | 27.2 | 4 | 21.3 | 100 | 20.3 | 39 | 27.1 | 2 |
| | | 24.04 | 69 | 27.6 | 4 | 22.6 | 14 | 20.8 | 26 | 27.9 | 2 |
| | | 24.835 | 24 | 28.7 | 7 | 23.0 | 9 | 21.6 | 100 | | |
| | | 25.22 | 13 | 29.7 | 5 | 24.5 | 38 | 22.0 | 15 | | |
| | | 26.39 | 13 | | | 25.5 | 28 | 22.5 | 12 | | |
| | | 27.385 | 13 | | | 25.6 | 25 | 22.9 | 12 | | |
| | | 28.165 | 1 | | | 26.7 | 12 | 23.6 | 14 | | |
| | | | | | | 28.4 | 9 | 24.6 | 24 | | |
| | | | | | | | | 25.0 | 64 | | |
| | | | | | | | | 25.6 | 17 | | |
| | | | | | | | | 26.1 | 28 | | |
| | | | | | | | | 26.5 | 24 | | |
| | | | | | | | | 27.1 | 7 | | |
| | | | | | | | | 29.0 | 12 | | |

*I/Iₒ relative intensity, Iₒ the most intensive signal

TABLE 4
Solid phase NMR data
Chemical shift (ppm)

| Polymorph | Polymorph | Polymorph | Polymorph | Polymorph |
|---|---|---|---|---|
| (IA) | (IB) | (IC) | (ID) | (IDb) |
| 175.0 | 175.4 | 176.6 | 176.1 | 174.8 |
| 156.3 | 155.6 | 157.0 | 157.5 | 156.8 |
| 151.4 | 151.7 | 153.8 | 150.0 | 153.4 |
| 148.6 | 149.4 | 150.9 | 138.7 | 151.9 |
| 135.9 | 135.9 | 149.3 | 137.4 | 150.2 |
| 129.6 | 129.4 | 136.9 | 131.7 | 148.4 |
| 124.4 | 125.0 | 135.5 | 130.0 | 137.8 |
| 119.4 | 119.3 | 127.7 | 123.3 | 131.4 |
| 111.7 | 112.1 | 126.5 | 121.2 | 129.3 |
| 103.3 | 103.3 | 1215 | 120.1 | 125.2 |
| 86.1 | 85.9 | 119.1 | 118.3 | 120.4 |
| 55.6 | 56.6 | 112.8 | 112.9 | 119.0 |
| 51.5 | 52.5 | 111.0 | 111.4 | 117.1 |
| 36.2 | 36.5 | 104.1 | 106.3 | 112.2 |
| 32.5 | 32.5 | 87.9 | 104.0 | 110.2 |
| 25.3 | 26.0 | 56.1 | 87.1 | 105.8 |
| 14.3 | 16.8 | 53.4 | 57.0 | 102.6 |
| 9.1 | 13.9 | 44.0 | 53.2 | 85.6 |
| | 11.5 | 40.4 | 52.0 | 55.3 |
| | 7.9 | 40.4 | 46.7 | 52.9 |
| | | 36.0 | 40.8 | 50.3 |
| | | 32.4 | 36.1 | 45.3 |
| | | 25.6 | 31.9 | 40.1 |
| | | 14.8 | 25.7 | 36.0 |
| | | 11.4 | 17.4 | 31.5 |
| | | | 16.0 | 24.7 |
| | | | 13.6 | 15.1 |
| | | | 11.1 | 13.8 |
| | | | | 11.7 |
| | | | | 10.6 |
| | | | | 9.3 |

TABLE 5
Thermoanalytical characteristics

| | Differential Scanning | | | Thermo- | |
|---|---|---|---|---|---|
| | Calorimetry (DSC) | | | gravimetry | |
| | | Tem- | | (TG) | |
| Poly- | | perature of | | Tem- | Loss of |
| morph, | | appearance | Enthalpy | perature | weight |
| Solvate | DSC peak | (° C.) | J/g | range | (%) |
| (IA) | Sharp Endoterm | 229.6 | −93.1 | | |
| (IB) | Sharp endoterm | 229.3 | −95.0 | | |
| (IC) | Endoterm- | 212.3 | — | | |
| | exoterm | | | | |
| | Sharp endoterm | 230.2 | — | | |
| (ID) | Sharp endoterm | 213.5 | — | | |
| | Sharp endoterm | 222.5 | — | | |
| (IDb) | Sharp endoterm | 224.6 | −88.5 | | |
| (IE) | Broad endoterm | 129.1 | −25.6 | | |
| | Broad exoterm | 180.8 | 71.2 | | |
| | Sharp endoterm | 229.4 | −94.1 | | |
| (IF) | Endoterm- | 167.4 | — | | |
| | exoterm | | | | |
| | Sharp endoterm | 230.4 | −94.1 | | |
| (IG) | Broad endoterm | 80-140 | — | 25-140 | 25.8% |
| | Broad exoterm | 179.3 | 52.5 | | |
| | Sharp endoterm | 229.7 | — | | |

1. Compound of formula: 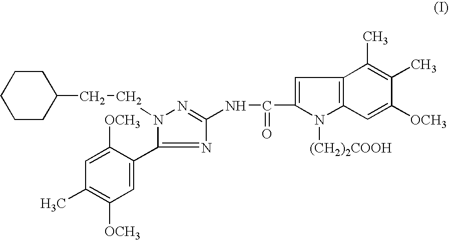
its solvates, hydrates, polymorphs and pharmaceutically acceptable salts:

2. Compound according to claim 1 in potassium salt form.

3. Salts of the 3-aminotriazole derivative of the formula (I) and of its polymorphic and solvate (pseudopolymorphic) forms, given with ethanolamine of the formula (A): HO—(CH₂)₂—NH₂, or diethanolamine of the formula (B): HO—(CH₂)₂—NH—(CH₂)₂—OH, or diethylamine of the formula (C): 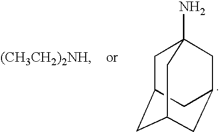
adamantanamine of the formula (D):

4. 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid ethanolamine salt.

5. Process for the preparation of compound of any of claim 1 to 4 characterized in that:
the compound of formula: 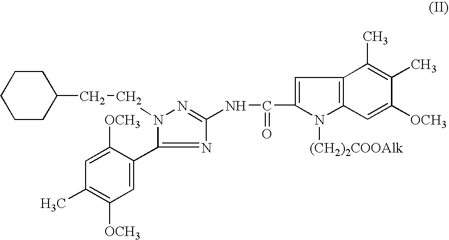
is hydrolysed;
if desired, the acid of formula (I) thus obtained is converted into its solvates, hydrates, polymorphs or pharmaceutically acceptable salts.

6. Process according to claim 5 for the preparation of the salts of the acid of formula (I) and of its polymorphic and solvate (pseudopolymorphic) forms, given with ethanolamine, diethanolamine, ethylamine, or with adamantanamine, which comprises reacting the acid of formula (I) or its polymorphic or solvate (pseudopolymorphic) forms with
ethanolamine of the formula (A), or
diethanolamine of the formula (B), or
diethylamine of the formula (C), or
adamantanamine of the formula (D).

7. The process as defined in claim 6 which comprises applying the compounds of formulae (A), (B), (C) or (D) in excess, preferably in a molar excess of 1.0-1.2.

8. The process as defined in claims 6 and 7 which comprises carrying out the reaction in a polar solvent, preferably in ethanol, acetone, or ethyl acetate.

9. Medicament characterized in that it comprises a compound according to anyone of claims 1 to 4.

10. Pharmaceutical compositions comprising as active principle a compound according to any one of claims 1 to 4.

11. Pharmaceutical composition according to claim 10 characterized in that it contains the active principle 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid potassium salt.

12. Pharmaceutical composition according to claim 10 characterized in that it contains the active principle 3-[2-[[[1-(2-cyclohexylethyl)-5-(2,5-dimethoxy-4-methylphenyl)-1H-1,2,4-triazol-3-yl]amino]carbonyl]-6-methoxy-4,5-dimethyl-1H-indol-1-yl]propionic acid ethanolamine salt.

13. Use of a compound according to any one of claims 1 to 4 for preparing medicaments intended for combating diseases whose treatment necessitates stimulation of cholecystokinin CCK₁ receptors.

14. Use of a compound according to any one of claims 1 to 4 for preparing medicaments intended for treating obesity.
